(19) United States
(12) Patent Application Publication
(10) Pub. No.: US 2023/0381300 A1
(43) Pub. Date: Nov. 30, 2023

(54) METHOD FOR MANUFACTURING HEK293 CELL LINE, METHOD FOR MANUFACTURING EB-VLPs AND COMPOSITION COMPRISING SAID EB-VLPs

(71) Applicant: Helmholtz Zentrum München Deutsches Forschungszentrum für Gesundheit und Umwelt (GmbH), Neuherberg (DE)

(72) Inventors: Wolfgang Hammerschmidt, Munich (DE); Dagmar Pich, Munich (DE)

(21) Appl. No.: 18/032,764

(22) PCT Filed: Oct. 20, 2021
(86) PCT No.: PCT/EP2021/079044

§ 371 Date: Apr. 19, 2023

(30) Foreign Application Priority Data
Oct. 20, 2020 (LU) 102132

(51) Int. Cl. A61K 39/245; C12N 7/00

(52) U.S. Cl. CPC A61K 39/245; C12N 7/00; C12N 2710/16223; C12N 2710/16222; C12N 2710/16234; C12N 2710/16252; A61K 2039/5258

(57) ABSTRACT

The invention provides a method for manufacturing a HEK293 cell line, which is capable of producing Epstein-Barr virus-like particles (EB-VLPs), as well as the HEK293 cell line obtainable by said method. The invention is further directed to a method for manufacturing EB-VLPs and a composition comprising EB-VLPs obtainable by said method for manufacturing EB-VLPs. Additionally, the invention provides a kit comprising EB-VLPs generated according to the method for manufacturing EB-VLPs. Further, the invention relates to a method for manufacturing a vaccine as well as the vaccine containing EB-VLPs obtainable by said method for manufacturing EB-VLPs.

### TECHNICAL FIELD OF THE INVENTION

The present invention provides a method for manufacturing a HEK293 cell line, which is capable of producing Epstein-Barr virus-like particles (EB-VLPs), as well as the HEK293 cell line obtainable by said method. The invention is further directed to a method for manufacturing EB-VLPs and a composition comprising EB-VLPs obtainable by said method for manufacturing EB-VLPs. Additionally, the invention provides a kit comprising EB-VLPs generated according to the method for manufacturing EB-VLPs. Further, the invention relates to a method for manufacturing a vaccine as well as the vaccine containing EB-VLPs obtainable by said method for manufacturing EB-VLPs.

### BACKGROUND ART

Epstein-Barr-Virus (EBV) is a ubiquitous human herpes virus that infects over 90% of the population world-wide with a life-long persistence in its human host. In most cases, primary infection occurs during early childhood and is usually asymptomatic. In contrast, if infection is delayed and happens during adolescence or adulthood, it is regularly symptomatic, causing a benign, lymphoproliferative syndrome termed infectious mononucleosis (IM) in up to 50% of cases. Although the disease is normally self-limiting, prolonged forms of IM, post-infection chronic fatigue or chronic active EBV infection (CAEBV) with fatal outcome have been reported. Clinical apparent IM has also been found to significantly increase the risk to develop Hodgkin disease and other type of lymphoma later in life. Similarily, IM is also a risk factor for Multiple Sclerosis later in life. In addition, EBV is causally associated with a heterogeneous group of malignant diseases such as nasopharyngeal carcinoma, gastric carcinoma, and various types of lymphoma, so that the WHO classifies EBV as a class I carcinogen.

An effective vaccine to prevent and treat infectious diseases caused by Epstein-Barr virus (EBV) is a long-standing issue in the field of infection medicine to control this herpes virus, wherein several different approaches have been tested, but still new concepts are required.

Towards this end, cell lines are used to synthesize the drug substance, which are virus-like particles (VLPs) to evoke broad, potent and effective immune responses against a plethora of viral antigens to prevent and treat infection diseases induced by this pathogen. VLPs are enveloped vesicles that contain about 50 different viral proteins, such as glycoproteins exposed on the vesicles' membrane, various tegument proteins and the viral capsid with its components contained in the lumen of the VLPs. Producer cells carry the genetic viral information in a genetically stable, so-called latent form and they release VLPs upon induction of the lytic, productive phase of the virus.

This standard approach to use VLPs is functional, which is, for example, described in Pich et al., 2019 and in Klinke et al., 2014. It is also a technical aspect in the patent application WO 2017/148928 A1 (Means and methods for treating herpesvirus infection) and EP 2608806 B1 (Epstein-Barr virus vaccine, see WO 2012/025603 A1).

The present invention aims at and addresses these needs described above. The inventors of the present invention addresses these needs by a method for manufacturing a HEK293 cell line, which is capable of producing Epstein-Barr virus-like particles (EB-VLPs), a method for manufacturing EB-VLPs, a composition comprising EB-VLPs, obtainable by said method for manufacturing EB-VLPs, and a vaccine containing EB-VLPs, obtainable by the method for manufacturing EB-VLPs, as the drug substance to evoke broad, potent and effective immune responses against a plethora of viral antigens to prevent and treat infection diseases induced by this pathogen.

### SUMMARY OF THE INVENTION

The above mentioned problems are solved by the subject-matter as defined in the claims and as defined herein.

The present invention provides in one aspect a method for manufacturing a HEK293 cell line, which is capable of producing Epstein-Barr virus-like particles (EB-VLPs), comprising
(a) introducing a vector comprising the EBV genome into said cell line, said vector being capable of being propagated both in a prokaryotic and eukaryotic host cell and being capable of autonomously replicating in said cell line; and
(b) removing from said vector nucleotide sequences required for its propagation in a prokaryotic host cell.

The present invention further provides a HEK293 cell line obtainable by the method of the present invention. In one embodiment, for said HEK293 cell line according to the present invention, the vector is free of non-viral sequences, except for a nucleotide sequence enabling the vector to be selectioned in the cell line. In one embodiment, said HEK293 cell line comprises an EBV gene encoding a polypeptide involved in the induction of the lytic cycle. In one preferred embodiment of said HEK293 cell line, said polypeptide involved in the induction of the lytic cycle is BZLF1, BRLF1, BMRF1, BMLF1, BALF2, BALF5, BGLF2, BHRF1, BALF4, BDLF3, or any combination thereof. In one further preferred embodiment of said HEK293 cell line, said polypeptide involved in the induction of the lytic cycle is a fusion protein between BZLF1 and an estrogen receptor.

In another aspect, the present invention provides a method for manufacturing EB-VLPs, comprising
(a) culturing the HEK293 cell line as described herein;
(b) inducing the lytic cycle;
(c) obtaining EB-VLPs; and optionally
(d) purifying said EB-VLPs.

The present invention further provides a composition comprising EB-VLPs obtainable by the method for manufacturing EB-VLPs as described herein. The composition according to the present invention may be for use as a vaccine. Further, the composition according to the present invention may be for use in the treatment and/or prevention of a disease. In one embodiment, the composition of the present invention comprises EB-VLPs and extracellular vesicles (EVs) in a ratio of 2:1 or greater, preferably of 3:1 or greater, more preferably of 5:1 or greater, even more preferably of 10:1 or greater.

Additionally, the present invention provides a kit comprising EB-VLPs generated according to the method for manufacturing EB-VLPs as described herein.

Further is provided by the present invention a method for the manufacturing of a vaccine, comprising the steps of the method for manufacturing EB-VLPs as described herein and the further step of formulating the EB-VLPs as a vaccine.

In another aspect, the present invention provides a vaccine containing EB-VLPs obtainable by the method for manufacturing EB-VLPs as described herein.

### BRIEF DESCRIPTION OF THE DRAWINGS

FIG. 1 shows the generation of EB-VLP producer cell lines and two modes of inducing EB-VLP production. FIG. 1A shows that the entire EBV genome (circle with dashed line) was cloned onto a prokaryotic backbone, a mini-F′ derived BACmid, to allow its propagation and genetic modification in E. coli cells (small ovoid elements in FIG. 1A) as described in the prior art (Delecluse et al., 1998). A viral gene (or a cis-acting DNA element) is deleted in E. coli to abolish the packaging of EBV's genomic DNA into viral particles (not shown here). Additional viral genes are eliminated by genetic modifications to enhance safety and efficacy of the vaccine product (not shown here). The mutated EBV genomic DNA (circle with continuous line) is isolated from E. coli, purified, and stably introduced into HEK293 cells, where it is maintained as extrachromosomal copies under selection. FIG. 1B shows standard techniques of EB-VLP production and release according to the prior art: HEK293 producer cells, which carry multiple nuclear copies of the mutated EBV genome (circle with continuous line) were established. To induce EB-VLP production and release, the cells were transiently transduced with plasmid DNA of an expression vector encoding the viral BZLF1 gene. For 4 days, the cells release EB-VLPs into the cell culture supernatant from which they are harvested. FIG. 1C shows the process of EB-VLP production and release according to the present invention. HEK293 producer cells, which carry multiple nuclear copies of the mutated EBV genome as in FIG. 1B were stably transduced with chromosomally integrated retroviral vector copies encoding the hormone-regulated conditional BZLF1 allele (not shown here). Upon addition of 4-hydroxy-tamoxifen (4-HO-TAM) the cells release EB-VLPs into the cell culture supernatant. The physical concentration of VLPs is indicated as measured by nanoparticle tracking analysis (NTA) four days after the addition of tamoxifen.

FIG. 2 shows the schematic overview of the relevant parts of the genetic composition of two EB-VLP producer genomes, particularly the genetic composition of the two EB-VLP producer genomes contained in 6507 and 87H7 cells. Shown are viral cis-acting elements (oriP, oriLyt, TR), several selected viral latent genes, their exons and promoters (arrows) together with micro RNAs of the BART cluster (miRNA BART). The viral genes BNLF1 (encoding the LMP1 oncoprotein) and miRNA BART are disabled or deleted as indicated. FIG. 2A shows a part of the genetic composition of the p6507.8 EB-VLP genome (SEQ ID NO: 20, shows nucleotide residues/coordinates #152,000-#169,676) contained in 6507 EV-VLP producer cells. The map highlights the mini-F′ plasmid backbone, the gfp gene and the gene hygromycin B phospho-transferase. Together, these elements make up the prokaryotic backbone, which encompasses 9,873 bps in size. FIG. 2B shows the genetic composition of the p6507.GFPneg.5 (Cre5) EB-VLP genome contained in 87H7 EB-VLP producer cells. Compared with the genetic composition of p6507.8 in FIG. 2A, only the gene encoding resistance against puromycin (puromycin N-acetyl-transferase) is maintained, but the prokaryotic backbone is completely deleted, meaning that the nucleotide sequences required for its propagation in a prokaryotic host cell is completely deleted.

FIG. 3 shows comparative analyses of EB-VLPs obtained with the standard technique versus the EB-VLPs obtained by the method of the present invention with VLP induction. FIG. 3A shows a schematic overview of the method to purify and analyze VLPs. EB-VLP producer cells were induced as shown in FIG. 1B or FIG. 10. The cell culture supernatants were collected after four days and purified by low and high speed centrifugations to remove cells and subcellular particles. Subsequently, the purified VLPs were stained with cell-trace yellow (CTY). Cell trace yellow is a cell-permeant amine-reactive fluorescent molecule, which enters cells or vesicles such as VLPs and extracellular vesicles (EVs) by diffusion through the membrane. Upon entry, the molecule is converted by esterases contained in the lumen of VLPs or EVs. The activated molecule covalently binds to amine groups in proteins, resulting in long-term dye retention within the lumen of the VLPs or EVs (cell trace yellow, CTY, is a product by Thermo Fisher). Staining of EVs and VLPs also included incubation with fluorochrome-coupled antibodies directed against the abundant viral glycoprotein gp350 present on the surface of VLPs (but absent on EVs) or an irrelevant antibody as a control. The EV/VLP preparations were further purified using iodixanol flotation density gradient centrifugation as described (Albanese et al., 2020). After ultracentrifugation, fractions known to contain both VLPs and EVs were collected and analyzed by flow cytometry using a Beckman Coulter Cytoflex instrument.

FIG. 3B shows representative results from flow cytometry analysis with VLP preparations stained with CTY and a control antibody coupled to APC or a gp350 specific APC-coupled antibody. The first, second and fifth panels of FIG. 3B show overall events recorded by the instrument, the CTY stained EV/VLP populations are marked and gated. (The ungated remaining events stem from background signals of the instrument, which are due to its extreme settings to detect the scatter of EVs or VLPs with an approximate diameter of 100 to 200 nm, only.) The third, fourth and sixth panels of FIG. 3B show the analysis of CTY stained EV/VLPs as gated above (x-axis) versus staining with control (third panel) and gp350-specific (fourth and sixth panels) antibodies coupled with APC (y-axis) as indicated. The percentages of gp350 positive and gp350 negative events per gate are provided in the quadrants. FIG. 3C shows flow cytometric analysis of VLP preparations as described in FIG. 3B of gradient fractions, as shown schematically in FIG. 3A. EB-VLP preparations stem from 87H7 or 6507 cells as indicated. The y-axis depicts the percentage of gp350-positive VLPs, the x-axis indicates the different fractions from the top of the gradient as in FIG. 3A. FIG. 3D, left panel, shows quantitative analysis of different VLP preparations with respect to their VLP/EV ratios after flow cytometry as in FIGS. 3B and 3C. The y-axis provides the percentage of gp350-positive VLPs from three independent preparations. TAM is a VLP preparation obtained by the method of the present invention as outlined in FIG. 10. The VLP preparations indicated “BZLF1, exp. 1” and “BZLF1, exp. 2” are single experiments and were obtained using the standard protocol by transient transfection of an expression plasmid encoding the viral BZLF1 gene as shown in FIG. 1B. These three plotted results were obtained from 87H7 EB-VLP producer cells, the fourth result stems from 6507 EB-VLP producer cells as indicated. TAM indicates induction with tamoxifen. FIG. 3D, right panel, shows relative quantitation of VLPs contained in the ‘purified VLP’ preparations as indicated in FIG. 3A prior to their further purification. The preparations were quantified using an established assay (Elijah cell binding assay) in which Elijah cells, a transformed human B-cell line (Baker et al., 1998), were incubated with different volumes of the VLP preparations. The incubated cells were washed and VLPs, which bind to the cells' surface, were stained with an APC-coupled gp350-specific antibody. The cells were subsequently analyzed by conventional flow cytometry together with a standard with a known concentration of infectious EBV particles that served as reference. The concentrations of VLPs in the four preparations were expressed as ‘green Raji units’ (GRU) equivalents per ml purified supernatant. VLP preparations obtained with the newly invented method contained substantially more VLPs (TAM) compared with two VLP preparations indicated “BZLF1, exp. 1” and “BZLF1, exp. 2” obtained by using the standard method of VLP induction. Three plotted results stem from EB-VLP preparations derived from 87H7 EB-VLP producer cells, the forth result stems from 6507 EB-VLP producer cells as indicated. FIG. 3E shows a panel that summarizes results from measuring the concentration of physical particles in the range of 100 to 200 nm by nanoparticle tracking analysis (NTA). The samples were taken from unconcentrated non-purified supernatants of tamoxifen induced 87H7 or 6507 EB-VLP producer cells as indicated four days post induction. The graph summarizes results from three independent VLP-containing supernatants from each producer cell line.

FIG. 4 shows the strategy how to remove the entire “prokaryotic backbone” (as defined herein) of the EB-VLP producer genome (see also Example 1), such that it can still be maintained stably in HEK293 cells, e.g. the sequences flanked by lox71 and lox66 sites were deleted upon transient expression of CRE. Shown are elements and genes in the following order (p6507.8 EB-VLP genome from nucleotide residues #152,000 to #169,676, SEQ ID NO: 20): lox71, SV40 polyadenylation signal, hygromycin B phosphotransferase (HPH), SV40 early promoter/enhancer, SV40 polyadenylation signal sequence, SV40 polyadenylation signal sequence, enhanced green fluorescent protein, gene: egfp, HCMV immediate-early promoter, F factor ori fragment, protein B (parB), protein A (parA), chloramphenicol acetyl-transferase, lox66, SV40 polyadenylation signal, puromycin N-acetyl-transferase, HCMV immediate-early promoter, LF3, OriLyt, lytic origin of replication, DR right, BILF1 reading frame (the sequence of the part of p6507.8 shown in FIG. 4 is provided as SEQ ID NO: 20).

FIG. 5A schematically shows a part of the parental EBV genome ΔmiR (4027), as described by Pich et al. (2010). The part spans nucleotide coordinates #176,332 to #179,740 of ΔmiR (4027) (SEQ ID NO: 21) together with relevant genes or exons of genes. The viral gene BNLF1 (SEQ ID NO: 8) encodes the oncoprotein LMP1 (SEQ ID NO: 5) and consists of three exons, a to c (SEQ ID NOs: 9, 10 and 11). FIG. 5B schematically shows the corresponding part of the EB-VLP producer genome p6507.8 from nucleotide coordinates #183,075 to #185,171 (SEQ ID NO: 22). In it, all three exons of BNLF1 (SEQ ID NOs: 9, 10 and 11) are deleted as indicated by the dashed line. The deletion in between nucleotide coordinates #184,739 and #184,740 in p6507.8 corresponds to the nucleotide coordinates #178,999 to #179,309 in ΔmiR (4027).

### DETAILED DESCRIPTION OF THE INVENTION

The invention provides in a first aspect a method for manufacturing a HEK293 cell line, which is capable of producing Epstein-Barr virus-like particles (EB-VLPs), comprising
(a) introducing a vector comprising the EBV genome into said cell line, said vector being capable of being propagated both in a prokaryotic and eukaryotic host cell and being capable of autonomously replicating in said cell line; and
(b) removing from said vector nucleotide sequences required for its propagation in a prokaryotic host cell.

The term “HEK293 cell line”, as used herein and in the context of the present invention, means a specific cell line, originally derived from human embryonic kidney cells, grown in tissue culture. HEK 293 cells have been widely used in cell biology research for many years, because of their reliable growth and propensity for transfection. They are also used by the biotechnology industry to produce therapeutic proteins and viruses for gene therapy. HEK293 cells have a complex karyotype, exhibiting two or more copies of each chromosome and with a modal chromosome number of 64. They are described as hypotriploid, containing less than three times the number of chromosomes of a haploid human gamete. Chromosomal abnormalities include a total of three copies of the X chromosome and four copies of chromosome 17 and chromosome 22.

The term “capable of” as used herein means that something is able to do, to perform the respective ability or property or to have all required means for being able to do so. For example, the respective HEK293 cell line obtained by the method of the present invention is able to produce Epstein-Barr virus-like particles.

The term “particle” as used in the present invention relates to a particulate conglomerate of EBV polypeptides and membrane lipids, while being devoid of EBV-DNA.

Virus-like particles (VLPs) are structurally similar to mature virions, but lack the viral genome. Therefore, VLPs are promising candidates for vaccination.

The term “EBV” as used herein relates to any wildtype, i.e. naturally occurring, EBV strain and is not restricted to one particular strain. Specifically, EBV type 1 and EBV type 2 strains are well-known in the art and have been extensively characterised. These two EBV-types differ largely in nuclear polypeptide genes that encode EBNA-LP, EBNA-2, EBNA-3A, EBNA-3B and EBNA-3C. Beyond differences relating to genes encoding polypeptides of the EBNA-family, the genomes of type 1 and 2 differ little. Type 1 is dominantly prevalent in developed world populations, whereas type 2 is also prevalent in equatorial Africa and New Guinea.

The term “vector” as used herein refers to a nucleic acid sequence into which one or more expression cassettes comprising a gene encoding the protein of interest may be inserted or cloned. Furthermore, the vector comprises the EBV genome. The genome of the Epstein-Barr virus consists of a linear, double-stranded DNA with a length of about 172,000 base pairs that encodes more than 80 different gene products. Preferably, the vector is a plasmid or viral vector.

The term “propagated” as used herein means to cause to multiply by any process of e.g. natural reproduction from the parent stock.

“Autonomously replicating” as used herein means that a vector is capable of duplication.

The term “prokaryotic/eukaryotic host cell” as used herein relates to any cell, which enables the vector comprising the EBV genome to be propagated in. Such an eukaryotic host cell is preferably a mammalian cell, more preferably a primate cell, even more preferably a human cell. Such a prokaryotic host cell is preferably a bacterial cell, more preferably a E. coli cell.

The vector comprising the EBV genome has been modified in step (b) of the method for manufacturing a HEK293 cell line such that from said vector nucleotide sequences required for its propagation in a prokaryotic host cell have been removed. Thus, the vector, respectively the EBV genome, has been modified in comparison to a recombinant wildtype EBV genome as described in Delecluse et al., 1998; Delecluse et al., 1999 or Pich et al., 2019. As outlined in the above sections, several wildtype EBV strains exist whose genetic make-up is well-known in the art. As is apparent from the above, the modified EBV genome is modified only with regard to sequences that are common to all EBV strains.

The term “polypeptide” refers to molecules consisting of more than 30 consecutive amino acids. Also envisaged in accordance with the present invention is to use “peptides”, i.e. molecules that comprise up to 30 amino acids, instead of polypeptides. Polypeptides may further form dimers, trimers and higher oligomers, i.e. consisting of more than one polypeptide molecule. Polypeptide molecules forming such dimers, trimers etc. may be identical or non-identical. The corresponding higher order structures are, consequently, termed homo- or heterodimers, homo- or heterotrimers etc. Homo- or heterodimers etc. also fall under the definition of the term “polypeptide”. The terms “polypeptide” and “protein” are used interchangeably herein and also refer to naturally modified polypeptides, wherein the modification is effected e.g. by glycosylation, acetylation, phosphorylation and the like. Such modifications are well known in the art. The terms “polynucleotide”, “nucleotide sequence”, “nucleic acid molecule” or “nucleic acid” are used interchangeably herein and refer to a polymeric form of nucleotides, which are usually linked from one deoxyribose or ribose to another. The term “polynucleotide” preferably includes single and double stranded forms of DNA. A nucleic acid molecule may include both sense and antisense strands of RNA (containing ribonucleotides), cDNA, genomic DNA, and synthetic forms and mixed polymers of the above.

Additionally, the term “removing from said vector nucleotide sequences required for its propagation in a prokaryotic host cell” is herein synonymously used with the term “removing the prokaryotic backbone” of the vector.

In one embodiment of the method for manufacturing a HEK293 cell line of the present invention, the vector comprising the EBV genome, in comparison to a wildtype EBV genome, is modified to lack one or more sequences that are required for the packaging of said wildtype EBV genome, is modified to lack one or more sequences encoding EBV polypeptides required for said packaging, is modified to lack one or more sequences encoding EBV polypeptides required for cleavage of viral DNA prior to said packaging, and/or is modified to comprise one or more sequences encoding EBV polypeptides, whose packaging capacity is disabled. Thus, of course, the removal of proteins that mediate the packaging and carry out the packaging directly disrupt the packaging. On the other hand, there are viral proteins that cut the newly replicated viral DNA into genome-sized pieces, which are then immediately packed in an empty viral capsid. The removal of such viral functions that cut the DNA also hampers packaging, simply because uncut DNA will not fit in a capsid.

Polypeptides termed “EBV polypeptides”, in accordance with the present invention, are polypeptides that are identical in their amino acid sequence to polypeptides of EBV. Further, the term “EBV polypeptide” comprises also polypeptides that are not identical to wildtype EBV strains as regards the sequence, but comprise proteins, which share at least (for each value) 99%, 98%, 97%, 96%, 95%, 90%, 85%, 80% and at least 75% identity in sequence to a wildtype EBV polypeptide. The degree of identity of polypeptide sequences can be calculated by well-known methods by the person skilled in the art and may comprise the automatic execution of algorithms effecting the alignment of sequence data and calculation of sequence homologies. The EBV polypeptides of the particle may originate from different EBV strains; preferably they originate from one strain.

The term “packaging” is well-known in the art with regard to virus assembly and relates to the process of introducing the linear EBV viral DNA into the virus particle during virus particle assembly. The packaging of EBV genomic DNA initiates at sequences (TR) that are directly repeated at both ends of said genomic DNA. Specifically, said modified EBV genome may lack one or more sequences that are required for packaging of a wildtype EBV genome, may lack one or more sequences encoding EBV polypeptides required for said packaging, and/or may lack one or more sequences encoding EBV polypeptides required for cleavage of viral DNA prior to said packaging. The term “required for packaging” means in accordance with the present invention that said one or more sequences are essential in packaging EBV DNA into a wildtype EBV particle. In other words, in the absence of said one or more sequences, the EBV DNA is not packaged into a wildtype EBV particle. The term “cleavage of viral DNA prior to packaging” means, as described above, the process mediated by viral proteins of cutting newly replicated viral DNA into genome-sized pieces, which are then immediately packed in an empty viral capsid. For this embodiment, it may be preferred that the one or more sequence encoding EBV polypeptides required for cleavage of viral DNA prior to said packaging, which is lacking, is BFRF1A. This lack of the gene BFRF1A prevents that the viral DNA is cut or is cut properly.

In one further embodiment of the method for manufacturing a HEK293 cell line of the present invention, the vector comprising the EBV genome, in comparison to a wildtype EBV genome, is modified to lack one or more sequences encoding EBV polypeptides that are required for B-cell transformation and/or is modified to comprise one or more sequences encoding EBV polypeptides, whose B-cell transformation capacity is disabled. It is preferred for said embodiment that the one or more EBV polypeptides required for B-cell transformation, which are lacking, are selected from the group consisting of LMP-1, LMP-2, EBNA-1, EBNA-2, EBNA-LP, EBNA-3A, EBNA-3B and EBNA-3C.

Disabling the capacity to transform B-cells can be achieved by methods such as modifying the polypeptide's domain that is functionally involved in the process of B-cell transformation. Said modifying can be achieved by methods such as, e.g., deletion of said functional domain or parts thereof, steric inhibition of said functional domain or the entire polypeptide, or substitution of one or more amino acids of said functional domain to the effect that function is compromised. When disabling a polypeptide's B-cell transformation capacity, its immunogenicity is to be maintained. The person skilled in the art is in the position to determine without further ado the antigenic regions of a protein and specific antigenic epitops within said regions by employing in silico as well as in vivo, in vitro or ex vivo routine experimental methods. After determination of said antigenic region he/she is in the position to choose a strategy that is suitable to maintain the immunogenicity of a polypeptide, while disabling its B-cell transformation capacity. This way, one can obtain functionally disabled, but immunogenic EBV polypeptides. For example, and in the case of transmembrane polypeptides having a B-cell transformation capacity such as, e.g., LMP-1, one can truncate said polypeptide by deleting only the transmembrane portion of said polypeptide, but preserving the extramembranous part of said polypeptide. The LMP-1 EBV polypeptide is a membrane spanning polypeptide that is required for B-cell transformation.

LMP-1 mimics a constitutively active CD40 receptor. LMP1 plays its central role during the latent phase of the virus in infected cells. Latently EBV-infected human B cells are transformed by the expression of a combination of latent EBV gene products. Among them is the key oncoprotein LMP1, which is encoded by the viral BNLF1 gene. In the latent phase of viral infection, however, the majority of the latently infected and transformed B cells does not produce and release viral progeny.

Epstein-Barr virus (EBV) latent membrane protein 2 (LMP-2) are two viral proteins of the Epstein-Barr virus. LMP2A/LMP2B are transmembrane proteins that act to block tyrosine kinase signaling. LMP2A is a transmembrane protein that inhibits normal B-cell signal transduction by mimicking an activated B-cell receptor (BCR). The N-terminus domain of LMP2A is tyrosine phosphorylated and associates with Src family protein tyrosine kinases (PTKs) as well as spleen tyrosine kinase (Syk). PTKs and Syk are associated with BCR signal transduction.

Epstein-Barr nuclear antigen 1 (EBNA-1) is a multifunctional, dimeric viral protein associated with Epstein-Barr virus (EBV). It is the only EBV protein found in all EBV-related malignancies. It is important in establishing and maintaining the altered state that cells take when infected with EBV. EBNA-1 has a glycine-alanine repeat sequence that separates the protein into amino- and carboxy-terminal domains. This sequence also seems to stabilize the protein, preventing proteasomal breakdown, as well as impairing antigen processing and MHC class I-restricted antigen presentation. This thereby inhibits the CD8-restricted cytotoxic T cell response against virus-infected cells.

The Epstein-Barr virus nuclear antigen 2 (EBNA-2) is one of the six EBV viral nuclear proteins expressed in latently infected B lymphocytes and is a transactivator protein. EBNA-2 is involved in the regulation of latent viral transcription and contributes to the immortalization of EBV infected cells. EBNA-2 acts as an adapter molecule that binds to cellular sequence-specific DNA-binding proteins, JK recombination signal-binding protein (RBP-JK), and PU.1 as well as working with multiple members of the RNA polymerase II transcription complex.

The Epstein-Barr virus (EBV) nuclear antigen leader protein (EBNA-LP) is the first viral latency-associated protein produced after EBV infection of resting B cells. It has been reported to enhance gene activation by the EBV protein EBNA2 in vitro.

The Epstein-Barr virus nuclear antigen 3 (EBNA-3) is a family of viral proteins associated with the Epstein-Barr virus. A typical EBV genome contains three such proteins: EBNA-3A (P12977, EBNA-3, BLRF3-BERF1), EBNA-3B (P03203, EBNA-4, BERF2A-BERF2B), EBNA-3C (P03204, EBNA-6, EBNA-4B, BERF3-BERF4). These genes also bind the host RBP-JK protein. EBNA-3C can recruit a ubiquitin ligase and has been shown to target cell-cycle regulators such as retinoblastoma protein (pRb).

Alternatively or in addition, the particle may be devoid of one or more EBV polypeptides that are required for B-cell transformation. Any of the above-mentioned EBV polypeptides required for B-cell transformation or EBV polypeptide combinations required for B-cell transformation may be lacking from the particle. In the case where one or more transforming polypeptides are lacking and at least one EBV polypeptide is disabled, it is understood that the one or more EBV polypeptide that is lacking cannot be disabled at the same time. In other words, if said one or more EBV polypeptides is disabled, it cannot be lacking at the same time. If a combination of EBV polypeptides is essential in B-cell transformation, a first member of the combination may be disabled, while a second member may be lacking.

In one embodiment of the method for manufacturing a HEK293 cell line of the present invention, the vector comprising the EBV genome, in comparison to a wildtype EBV genome, is modified to lack one or more sequences encoding EBV polypeptides that are required for inducing replication of an EBV and/or is modified to comprise one or more sequences encoding EBV polypeptides, whose capacity for inducing EBV replication is disabled. It is preferred for said embodiment that the one or more EBV polypeptides that are required for inducing replication of an EBV, which are lacking, or said one or more EBV polypeptides, whose capacity for inducing EBV replication is disabled, are selected from the group consisting of BZLF1, BRLF1, BMLF1 and any combination thereof. It is even more preferred for said embodiment, that the one or more EBV polypeptides that are required for inducing replication of an EBV, which are lacking, or said one or more EBV polypeptides, whose capacity for inducing EBV replication is disabled, is BZLF1.

BZLF1 (BamHI Z fragment leftward open reading frame 1), also known as Zta, ZEBRA, EB1, is an immediate-early viral gene of the Epstein-Barr virus (EBV) of the Herpes Virus Family, which induces cancers and infects primarily the B-cells of 95% of the human population. This gene (along with others) produces the expression of other EBV genes in other stages of disease progression, and is involved in converting the viral infection from the latent to the lytic form.

Epstein-Barr virus (EBV) immediate-early (IE) protein BRLF1 induces the lytic form of viral replication in most EBV-positive cell lines. BRLF1 is a transcriptional activator that binds directly to a GC-rich motif present in some EBV lytic gene promoters. However, BRLF1 activates transcription of the other IE protein, BZLF1.

Epstein-Barr virus immediate-early gene product, BMLF1, stimulates the preferred nuclear export of viral RNAs for their translation into the cytoplasm of the cell that supports EBV's lytic form of infection.

The term “inducing replication” of EBV means in accordance with the present invention the initiation of the process that ultimately leads to intracellular assembly of virus-like particles (VLPs) and egress of said VLPs as particles defined herein. The induction can be achieved, for example, by complementing the cell with said one or more EBV polypeptides that are absent from the host cell due to the deletion of said one or more sequences encoding EBV polypeptides that are required for inducing replication and/or the modification of said one or more sequences encoding EBV polypeptides resulting in their inability to induce EBV replication. Said complementation can be achieved e.g. by providing the cell with said deleted one or more sequences, e.g. on a plasmid (stably or transiently transfected), from which the missing EBV polypeptides can be expressed; or by providing the cell with one or more unmodified sequences encoding functional EBV polypeptides that are capable of inducing replication. The provision of said one or more DNA sequences can be effected by methods as described herein above and in the examples-section. Alternatively, said complementation can be achieved by providing the cell said one or more EBV polypeptides required for inducing replication of an EBV. The provision of said one or more polypeptides to the cell can be achieved by protein delivery methods that are well-known in the art and may involve the use of reagents such as, e.g., ProteoJuice (Merck), TurboFect (Fermentas) or Lipodin-Pro (Abbiotec).

In one further embodiment of the method for manufacturing a HEK293 cell line of the present invention, the vector comprising the EBV genome, in comparison to a wildtype EBV genome, is modified to lack one or more expressible gene(s) selected from the group consisting of the BFLF1 gene, the BBRF1 gene, the BGRF1 gene, the BDRF1 gene, the BALF3 gene, the BFRF1A gene, and the BFRF1 gene. The proteins of said genes are required for cleavage or packaging of viral DNA (i.e. an EBV genome or the at least one nucleic acid molecule) into procapsids of EBV. Accordingly, the at least one nucleic acid molecule or an EBV genome is not packaged into the procapsid of EBV if one or more of said genes are genetically modified such that said EBV protein is not expressed or non-functional and thus results in the production of the HEK293 cell line and the EB-VLPs of the present invention.

The BFLF1 gene is a member of the herpesviridae UL32 protein family and encodes the packaging protein UL32 homolog, playing a role in efficient localization of neo-synthesized capsids to nuclear replication compartments, thereby controlling cleavage and packaging of virus genomic DNA.

The BBRF1 gene encodes a portal protein, which forms a portal in the viral capsid through which viral DNA is translocated during DNA packaging.

The BGRF1 gene encodes the respective protein with the same name, also being involved in DNA packaging.

The BDRF1 gene encodes the protein tripartite terminase subunit 3 (TRM3), a component of the molecular motor that translocates viral genomic DNA into empty capsids during DNA packaging. It forms a tripartite terminase complex together with BALF3 (Tripartite terminase subunit 1, TRM1) and BFRF1A (Tripartite terminase subunit 2, TRM2) in the host cytoplasm. Once the complex reaches the host nucleus, it interacts with the capsid portal vertex. This portal forms a ring, in which genomic DNA is translocated into the capsid. TRM3 carries an RNase H-like nuclease activity that plays an important role for the cleavage of concatemeric viral DNA into unit length genomes. Said “terminase” is the enzyme complex that cuts the viral DNA within the terminal repeats (TR in FIG. 2), such that a single complete copy of unit length viral DNA, after it was channeled through the portal into the capsid, is severed and thus separted from the bulk of newly replicated viral DNA. The terminase subunits of EBV have been identified as BALF3, BDRF1, and BFRF1A by analysis of sequence homologies.

The BALF3 gene (Tripartite terminase subunit 1, TRM1) encodes the respective protein with the same name, also being involved in viral DNA packaging.

The gene BFRF1 encodes the protein nuclear egress protein 2 (NEC2), playing an essential role in virion nuclear egress, the first step of virion release from infected cell. Within the host nucleus, NEC1 interacts with the newly formed capsid through the vertexes and directs it to the inner nuclear membrane by associating with BFLF2 (NEC2). This induces the budding of the capsid at the inner nuclear membrane as well as its envelopment into the perinuclear space. There, the NEC1/NEC2 complex promotes the fusion of the enveloped capsid with the outer nuclear membrane and the subsequent release of the viral capsid into the cytoplasm, where it will reach the secondary budding sites in the host Golgi or trans-Golgi network.

The gene BFRF1A encodes the protein tripartite terminase subunit 2 (TRM2), also a component of the molecular motor that translocates viral genomic DNA in empty capsids during DNA packaging. It may also be described as “UL33 HSV-1 homolog” (HSV-1=herpes simplex virus 1) of EBV.

In one embodiment of the method for manufacturing a HEK293 cell line of the present invention, the vector comprising the EBV genome, in comparison to a wildtype EBV genome, is modified to lack one or more sequences encoding EBV polypeptides selected from the group consisting of the BNRF1 polypeptide, the BPLF1 polypeptide, the BGLF3 polypeptide, the BRRF2 polypeptide, the BKRF4 polypeptide and the BXLF1 polypeptide.

The BNRF1 polypeptide is also called major tegument protein, a tegument protein that plays a role in the inhibition of host intrinsic defenses to promote viral early gene activation.

The BPLF1 polypeptide is also called large tegument protein deneddylase. The large tegument protein plays multiple roles in the viral cycle. During viral entry, it remains associated with the capsid, while most of the tegument is detached and participates in the capsid transport toward the host nucleus. It plays a role in the routing of the capsid at the nuclear pore complex and subsequent uncoating. Within the host nucleus, it acts as a deneddylase and promotes the degradation of nuclear CRLs (cullin-RI NG ubiquitin ligases) and thereby stabilizes nuclear CRL substrates, while cytoplasmic CRLs remain unaffected. These modifications prevent the host cell cycle S-phase progression and create a favorable environment allowing efficient viral genome replication. It also participates later in the secondary envelopment of capsids. Further, it plays a linker role for the association of the outer viral tegument to the capsids together with the inner tegument protein.

The BGLF3 polypeptide belongs to the herpesviridae UL95 family.

The BRRF2 polypeptide is also called tegument protein BRRF2 and belongs to the lymphocryptovirus BRRF2 family.

The BKRF4 polypeptide is also called tegument protein BKRF4 and belongs to the lymphocryptovirus BKRF4 family.

The BXLF1 polypeptide is a thymidine kinase, catalyzing the transfer of the gamma-phospho group of ATP to thymidine to generate dTMP in the salvage pathway of pyrimidine synthesis. The dTMP serves as a substrate for DNA polymerase during viral DNA replication. It allows the virus to be reactivated and to grow in non-proliferative cells, lacking a high concentration of phosphorylated nucleic acid precursors.

The method for manufacturing a HEK293 cell line further comprises, in one preferred embodiment, that the vector comprising the EBV genome, in comparison to a wildtype EBV genome, is modified to lack one or more miRNAs, selected from the group consisting of miR-BHRF1-1, miR-BHRF1-2, miR-BHRF1-3, miR-BART1, miR-BART2, miR-BART3, miR-BART4, miR-BART5, miR-BART6, miR-BART7, miR-BART8, miR-BART9, miR-BART10, miR-BART11, miR-BART12, miR-BART13, miR-BART14, miR-BART15, miR-BART16, miR-BART17, miR-BART18, miR-BART19, miR-BART20, miR-BART21, and miR-BART22. In one further preferred embodiment of the method for manufacturing a HEK293 cell according to the present invention, the vector comprising the EBV genome, in comparison to a wildtype EBV genome, is modified to lack one or more miRNAs, as described in WO 2017/148928 A1. The mentioned miR-BHRF1-1, miR-BHRF1-2, miR-BHRF1-3, miR-BART1, miR-BART2, miR-BART3, miR-BART4, miR-BART5, miR-BART6, miR-BART7, miR-BART8, miR-BART9, miR-BART10, miR-BART11, miR-BART12, miR-BART13, miR-BART14, miR-BART15, miR-BART16, miR-BART17, miR-BART18, miR-BART19, miR-BART20, miR-BART21, and miR-BART22 are the 25 hairpin precursor RNAs of the EBV genome, from which the mature miRNAs are created in a further, final process step. In purely mathematical terms, two miRNAs can arise from one hairpin precursor RNA. So 25 precursor RNAs result in 50 mature miRNAs. However, some of these miRNAs are so unstable that they cannot be detected. In EBV infected cells there are therefore only 44 mature miRNAs. According to http://www.mirbase.org/textsearch.shtml?q=ebv&submit=submit, miR-BHRF1-1 is given as Accession Number MI0001064, miR-BHRF1-2 is given as Accession Number MI0001065, miR-BHRF1-3 is given as Accession Number MI0001066, miR-BART1 is given as Accession Number MI0001067, miR-BART2 is given as Accession Number MI0001068, miR-BART3 is given as Accession Number MI0003728, miR-BART4 is given as Accession Number MI0003726, miR-BART5 is given as Accession Number MI0003727, miR-BART6 is given as Accession Number MI0003728, miR-BART7 is given as Accession Number MI0003729, miR-BART8 is given as Accession Number MI0003730, miR-BART9 is given as Accession Number MI0003731, miR-BART10 is given as Accession Number MI0003732, miR-BART11 is given as Accession Number MI0003733, miR-BART12 is given as Accession Number MI0003734, miR-BART13 is given as Accession Number MI0003735, miR-BART14 is given as Accession Number MI0003736, miR-BART15 is given as Accession Number MI0004988, miR-BART16 is given as Accession Number MI0004989, miR-BART17 is given as Accession Number MI0004990, miR-BART18 is given as Accession Number MI0004991, miR-BART19 is given as Accession Number MI0004992, miR-BART20 is given as Accession Number MI0004993, miR-BART21 is given as Accession Number MI0010627, and miR-BART22 is given as Accession Number MI0010628.

The term “miRNA” as used herein relates to small non-coding single-stranded RNAs of about 21 to 25 nucleotides in length. The 5′-ends of miRNAs, the so-called seed sequences, recognize partially complementary mRNA targets usually within their 3′ untranslated regions and repress translation of these mRNAs. miRNA coding loci are first transcribed into longer primary miRNAs (pri-miRNAs) usually by RNA polymerase II. The RNase III enzyme Drosha then recognizes and cleaves the pri-miRNAs to liberate hairpin structures, usually 60 to 80 nt long, called pre-miRNAs, which are transported into cytoplasm and further processed by another RNase III enzyme named Dicer to produce RNA duplexes. The RNA duplexes associate with Argonaute (Ago) proteins, Dicer, and GW182 in RNA-induced silencing complexes (RISC), where they are unwound. Often, at this stage, one strand (the “star strand”) is degraded, while the other strand (mature miRNA) is retained. The RISC is guided by the miRNAs to specifically recognize and regulate target mRNAs. Thus, miRNAs are key regulators of a number of biological processes including developmental timing, differentiation and pattering, but also cellular proliferation, cell death, immune response, haematopoiesis, and cellular transformation or oncogenesis. One single miRNA may directly regulate the expression of hundreds of different mRNAs.

Forty-four microRNAs (miRNAs) (“mature” EBV miRNAs) and 25 hairpin precursors from which the mature EBV miRNAs are derived have been identified that are transcribed during latent infection from three clusters in the EBV genome. Two of the three clusters of miRNAs are made from the BamHI A rightward transcripts (BARTs), a set of alternatively spliced transcripts that are highly abundant in NPC, but have not been shown to produce a detectable protein. This study indicates that while the BART miRNAs are located in the first four introns of the transcripts, processing of the pre-miRNAs from the primary transcript occurs prior to completion of the splicing reaction. Additionally, production of the BART miRNAs correlates with accumulation of a spliced mRNA, in which exon 1 is joined directly to exon 3, suggesting that this form of the transcript may favor production of miRNAs.

The Epstein-Barr virus (EBV) miR-BHRF1 microRNA (miRNA) cluster has been shown to facilitate B-cell transformation and to promote the rapid growth of the resultant lymphoblastoid cell lines (LCLs).

In one embodiment of the method for manufacturing a HEK293 cell line according to the present invention, step (b) comprises modifying the vector to be free of non-viral sequences, except for a nucleotide sequence enabling the vector to be selectioned in the cell line. The term “to be free of non-viral sequences” as used herein means that the vector is devoid of sequences, which are not related to a virus or which have no viral origin.

In one further embodiment, the method for manufacturing a HEK293 cell line according to the present invention further comprises introducing an EBV gene encoding a polypeptide involved in the induction of the lytic cycle. It is preferred for this embodiment, that said polypeptide involved in the induction of the lytic cycle is BZLF1, BRLF1, BMRF1, BMLF1, BALF2, BALF5, BGLF2, BHRF1, BALF4, BDLF3, or any combination thereof. It is even more preferred for said embodiment, that said polypeptide involved in the induction of the lytic cycle is a fusion protein between BZLF1 and an estrogen receptor. Said fusion protein between BZLF1 and an estrogen receptor (ER) may have a nucleotide sequence as given herein as SEQ ID NO: 19 and a respective amino acid sequence as given herein as SEQ ID NO: 18.

The Epstein-Barr virus (EBV) BMRF1 promoter for early antigen (EA-D) is regulated by the EBV transactivators, BRLF1 and BZLF1, in a cell-specific manner and the gene encodes an early lytic protein that functions not only as the viral DNA polymerase processivity factor, but also as a transcriptional activator.

BALF2 is a major DNA-binding protein, playing several crucial roles in viral infection. It participates in the opening of the viral DNA origin to initiate replication by interacting with the origin-binding protein. It may disrupt loops, hairpins and other secondary structures present on ssDNA to reduce and eliminate pausing of viral DNA polymerase at specific sites during elongation. It promotes viral DNA recombination by performing strand-transfer, characterized by the ability to transfer a DNA strand from a linear duplex to a complementary single-stranded DNA circle. It can also catalyze the renaturation of complementary single strands. Additionally, it reorganizes the host cell nucleus, leading to the formation of pre-replicative sites and replication compartments. This process is driven by the protein, which can form double-helical filaments in the absence of DNA.

The BALF4 polypeptide is also called envelope glycoprotein B (gB), that forms spikes at the surface of the virion envelope. It is essential for the initial attachment to heparan sulfate moieties of the host cell surface proteoglycans. It is involved in fusion of viral and cellular membranes leading to the virus entry into the host cell. Following initial binding to its host receptors, membrane fusion is mediated by the fusion machinery composed at least of gB and the heterodimer gH/gL.

The BALF5 polypeptide is also called DNA polymerase catalytic subunit. It replicates viral genomic DNA in the late phase of lytic infection, producing long concatemeric DNA. The replication complex is composed of six viral proteins: the DNA polymerase, processivity factor, primase, primase-associated factor, helicase, and ssDNA-binding protein.

The BGLF2 polypeptide is a tegument protein, regulates the cellular AP-1 pathway and contributes to the synthesis of viral progeny.

The BHRF1 polypeptide is also called apoptosis regulator BHRF1. It prevents premature death of the host cell during virus production, which would otherwise reduce the amount of progeny virus. It acts as a host B-cell leukemia/lymphoma 2 (Bcl-2) homolog, and interacts with pro-apoptotic proteins to prevent mitochondria permeabilization, release of cytochrome c and subsequent apoptosis of the host cell.

The BDLF3 is also called glycoprotein BDLF3 and belongs to the Epstein-Barr virus BDLF3 protein family.

The term “estrogen receptor” as used herein may comprise any member of the estrogen receptor family, which are receptors that are activated by the hormone estrogen (1713-estradiol). Two classes of ER exist: nuclear estrogen receptors (ERα and ERβ), which are members of the nuclear receptor family of intracellular receptors, and membrane estrogen receptors (mERs) (GPER (GPR30), ER-X, and G_{q}-mER), which are mostly G protein-coupled receptors. Once activated by estrogen, the ER is able to translocate into the nucleus and bind to DNA to regulate the activity of different genes (i.e. it is a DNA-binding transcription factor). Thus, ER is a member of a superfamily of hormone-regulated transcription factors that stimulate gene expression in response to estrogens. The ability to switch ER functions from inactive to active state by simply adding ligand has illuminated several fundamental insights into eukaryotic transcriptional regulation. However, the answer to the important question of how this simple linear model eventually culminates in gene transcription lies with coregulator proteins.

The term “estrogen receptor” as used herein also comprises any member of the family of steroid hormone receptors, which is preferably the glucocorticoid receptor, the androgen receptor, the estrogen receptors α and β, the progesterone receptor, or the mineralocorticoid receptor.

The present invention also comprises the respective HEK293 cell line obtainable by any of the methods for manufacturing a HEK293 cell line as described herein. The above given definitions with regard to the method for manufacturing the HEK293 cell line also apply mutatis mutandis to the obtained HEK293 cell line.

In one embodiment of the HEK293 cell line according to the present invention, the vector is free of non-viral sequences, except for a nucleotide sequence enabling the vector to be selectioned in the cell line as defined above.

It is further preferred for the HEK293 cell line according to the present invention to comprise an EBV gene encoding a polypeptide involved in the induction of the lytic cycle, more preferably wherein said polypeptide involved in the induction of the lytic cycle is BZLF1, BRLF1, BMRF1, BMLF1, BALF2, BALF5, BGLF2, BHRF1, BALF4, BDLF3, or any combination thereof. More preferably, said polypeptide involved in the induction of the lytic cycle is a fusion protein between BZLF1 and an estrogen receptor (ER) as defined herein above. Said fusion protein between BZLF1 and an estrogen receptor (ER) may have a nucleotide sequence as given herein as SEQ ID NO: 19 and a respective amino acid sequence as given herein as SEQ ID NO: 18.

The present invention also provides a method for manufacturing EB-VLPs, comprising
(a) culturing the HEK293 cell line as described herein and as obtained by any of the methods for manufacturing according to the present invention;
(b) inducing the lytic cycle;
(c) obtaining EB-VLPs; and optionally
(d) purifying said EB-VLPs.

In one preferred embodiment, the present invention provides a method for manufacturing EB-VLPs, comprising
(a) culturing the HEK293 cell line as described herein and as obtained by any of the methods for manufacturing according to the present invention;
(b) inducing the lytic cycle;
(c) obtaining EB-VLPs; and
(d) purifying said EB-VLPs.

The term “culturing” as used herein relates to growing cells outside the organism in cell culture medium and is known by the person skilled in the art. Suitable cell culture media confer survival and replication by the cells and are commercially available. They may comprise nutrients, salts, growth factors, antibiotics, serum (e.g. fetal calf serum) and pH-indicators (e.g. phenol red). The term “culturing” is used in accordance with its accepted meaning in the art. Generally, cell culture methods, such as, for example, media constituents, marker choice and selection, cell quantification and isolation, are methods well-known in the art and described, for example, in “Practical Cell Culture Techniques”, Boulton et Baker (eds), Humana Press (1992); “Human Cell Culture Protocols”, Gareth E. Jones, Humana Press (1996), and exemplarily in the examples-section. Culture conditions vary from cell-type to cell-type and moreover, can result in different phenotypes being expressed for a particular cell-type. Generally, cells are grown and maintained at an appropriate temperature and gas mixture, i.e. typically 37° C., 5% CO₂, in growth media (a) as irrigating, transporting and diluting fluid, while maintaining intra- and extra-cellular osmotic balance, (b) that provides cells with water and certain bulk inorganic ions essential for normal cell metabolism, (c) which, combined with a carbohydrate, such as glucose, provides the principle energy source for cell metabolism, and (d), which provides a buffering system to maintain the medium within a physiologic pH range, i.e. cells are kept viable. The recipe of growth media varies greatly depending on cell-type and contains, for example, and without limitation, growth factors, nutrient components, glucose, buffers to maintain pH and antifungizides and -biotics. Methods for culturing and maintaining cells in culture are well-known in the art. Growth media and other cell culture related material as well as instructions and methods for successful culturing of cells can, for example, be obtained at Sigma-Aldrich or Invitrogen. The conditions to allow expression of the modified EBV genome correspond essentially to the general conditions described herein above. Modifications to enhance polypeptide expression from the EBV genome in the host cell are known to the person skilled in the art.

The term “inducing the lytic cycle” as used herein and in the present invention may comprise that the lytic phase of a Herpes virus is induced and maintained upon expression of certain herpes viral proteins, e.g. BZFL1, BRLF1 and BMLF1, in case of Epstein-Barr virus.

In one preferred embodiment of the method for manufacturing EB-VLPs, step (b) further comprises the addition of estrogen, tamoxifen or derivatives thereof. Estrogen and tamoxifen (C₂₆H₂₉NO, Mᵣ=371.5 g/mol) are well known to the person skilled in the art. As a preferred derivative of tamoxifen, 4-hydroxy-tamoxifen may be used.

In one embodiment of the method for manufacturing EB-VLPs, the step (c) of obtaining EB-VLPs may comprise inducing the synthesis of VLPs in the producer cell line and their release into the cell culture supernatant, by transiently transfection of the cells with a plasmid DNA that encodes a viral gene, e.g. BZLF1 as defined herein above. The expression of BZLF1 in the transfected cell line may induce the synthesis of the VLPs.

The term “obtaining” as used herein relates to isolating and/or purifying the EB-VLPs, preferably from the cell culture supernatant. Such isolation and/or purification steps are known to the person skilled in the art and encompass, for example, methods such as density gradient centrifugation, size-exclusion chromatography, tangential flow ultrafiltration, affinity chromatography, precipitation and, in case of EBV, the binding of EB-VLP to magnetic beads via anti-gp350 antibodies. In one preferred embodiment of the method for manufacturing EB-VLPs, step (d) comprises high speed centrifugation, ultracentrifugation, flotation density and/or gradient centrifugation. The further analyzing may be done by staining, preferably with a fluorescent molecule.

The term “transfection” is used in connection with the present invention according to the accepted meaning in the art, viz. the process of introducing nucleic acids into cells. Transfection can be achieved by a variety of methods such as, e.g. chemical-based methods, like calcium phosphate-mediated transfection or liposome-mediated transfection. Also non-chemical methods, like electroporation or sonoporation, or particle-based methods, such as gene-gun-mediated transfection or magnetofection as well as viral-mediated methods are known in the art.

The present invention further provides a composition comprising EB-VLPs obtainable by the method for manufacturing EB-VLPs as described herein and according to the present invention.

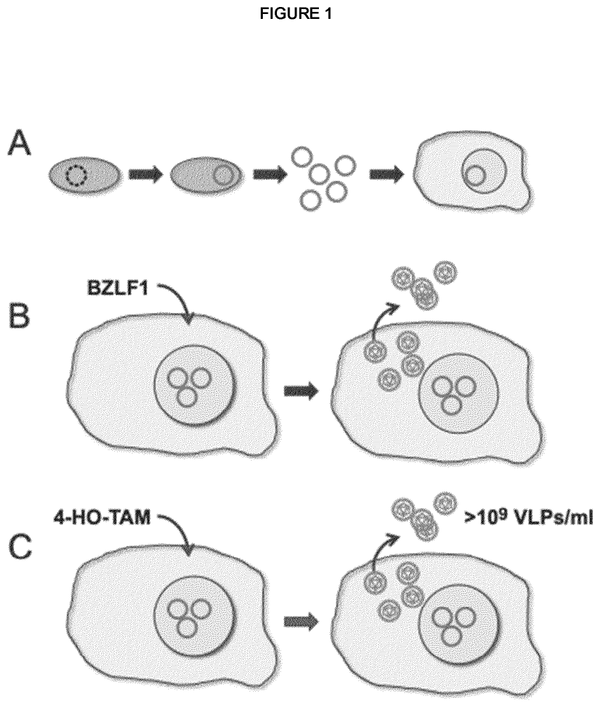

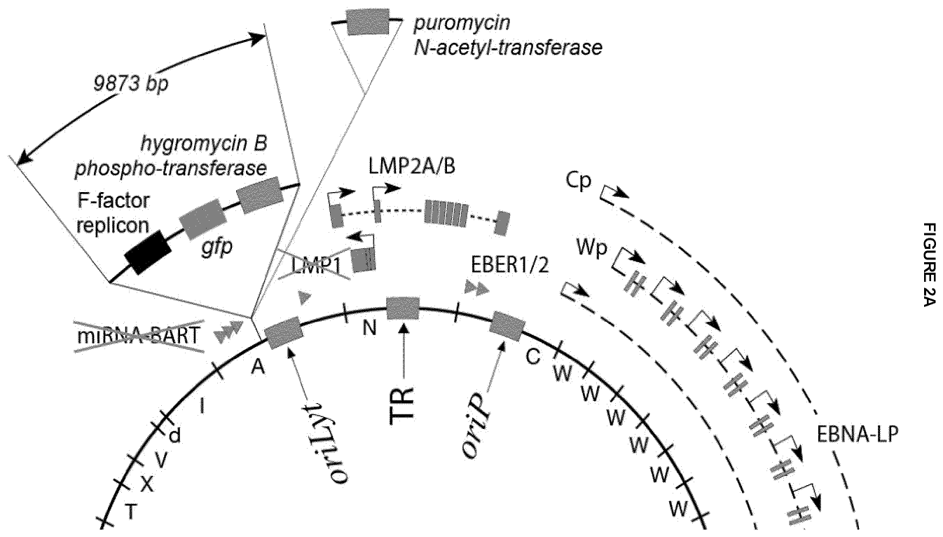

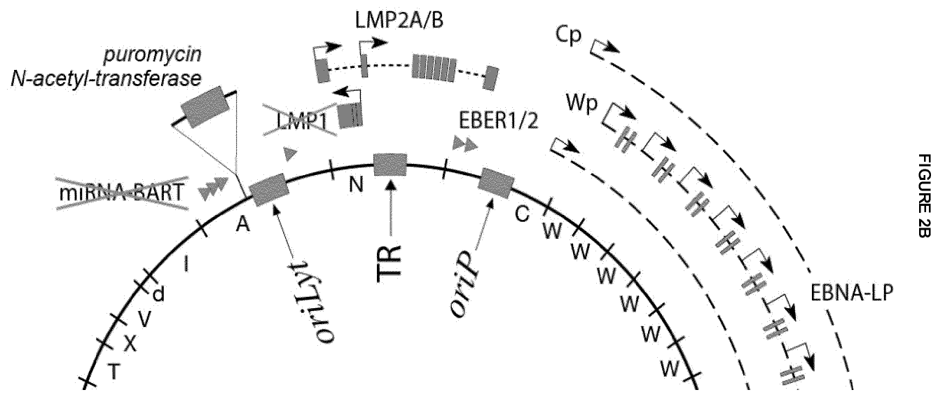

The approach schematically shown in FIG. 1B according to the prior art is disadvantageous regarding the purity and yield of a composition comprising EB-VLPs. This is because only a fraction of the cells take up DNA upon transient transfection of the BZLF1 expression plasmid. With this approach according to the prior art, up to 50% of the cells are transfected at best, but only transfected cells will support the synthesis and release of EB-VLPs into the cell culture medium. Non-transfected cells will be part of the cell culture as it is practically difficult to separate non-transfected cells from cells that are successfully transfected with the BZLF1 expression plasmid DNA. Non-transfected cells do not release EB-VLPs, but continue to release vesicles into the cell culture supernatant, so-called extracellular vesicles (EVs). The membranous EV particles are physically indistinguishable from EB-VLPs. The number of EVs and the number of EB-VLPs released from non-transfected and transfected EB-VLP producer cells, respectively, are similar, but only EB-VLPs constitute the drug substance that contains the viral antigens for immunization of the vaccinees. Practically, it is difficult or at least technically very demanding to enrich EB-VLPs and remove EVs from the cell culture supernatants to purify the vaccine drug product. From a regulatory point of view, EVs are adventitious materials that increase the level of host cell protein in the drug substance and bears a risk that needs to be minimized in the drug product. Thus, in one embodiment, the method for manufacturing EB-VLPs aims at minimizing the content of EVs.

In one further embodiment, the composition according to the present invention is for use as a vaccine.

The composition according to the present invention is preferably also for use in the treatment and/or prevention of a disease.

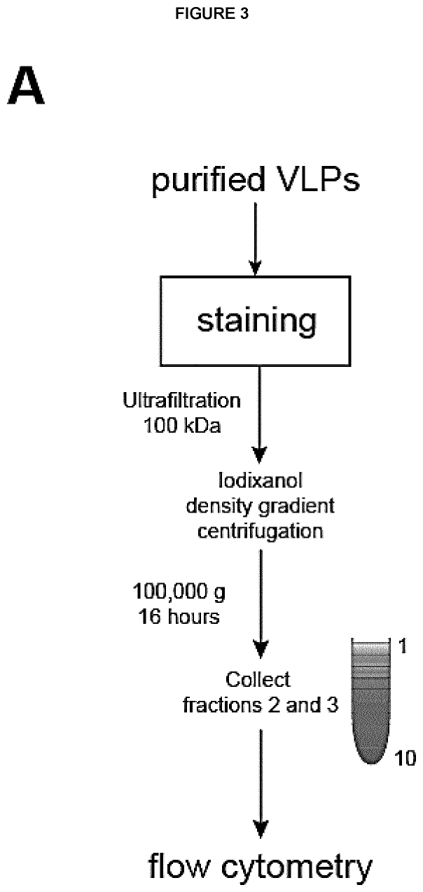

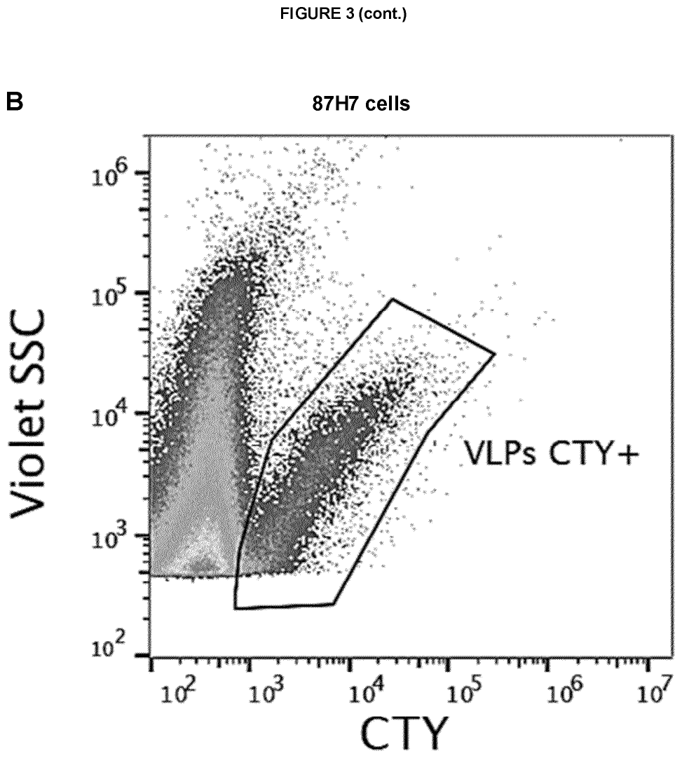

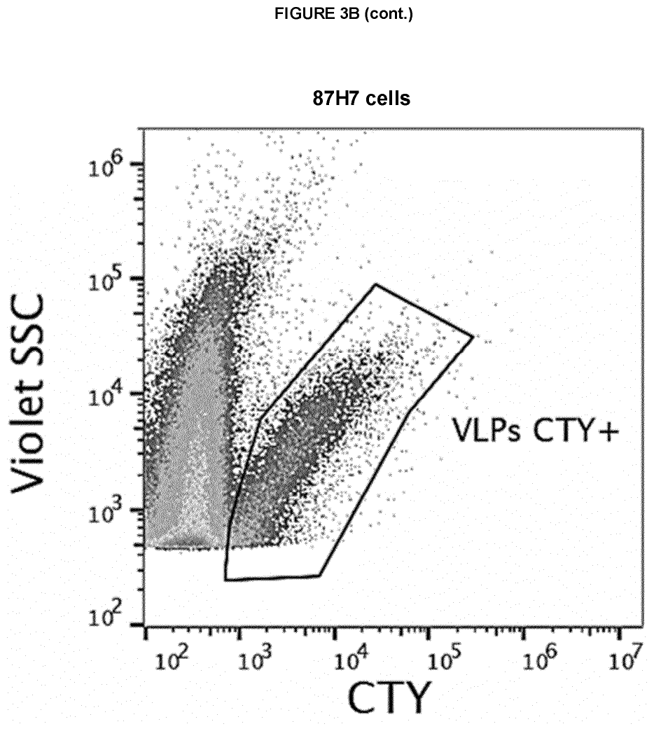

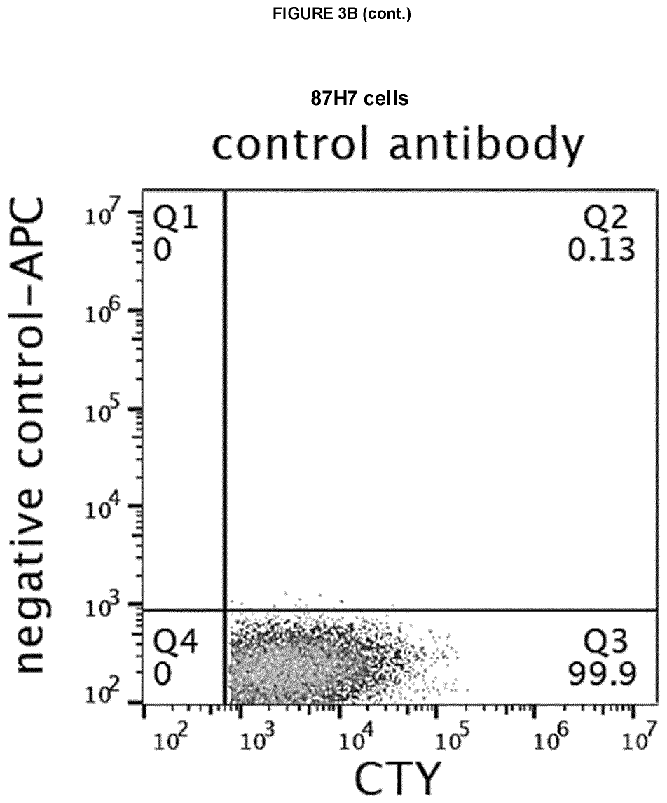

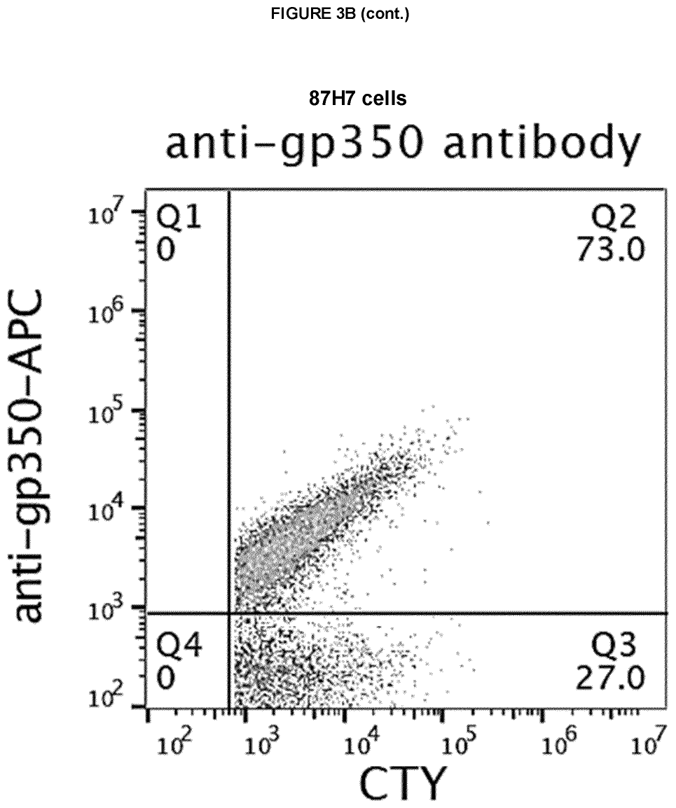

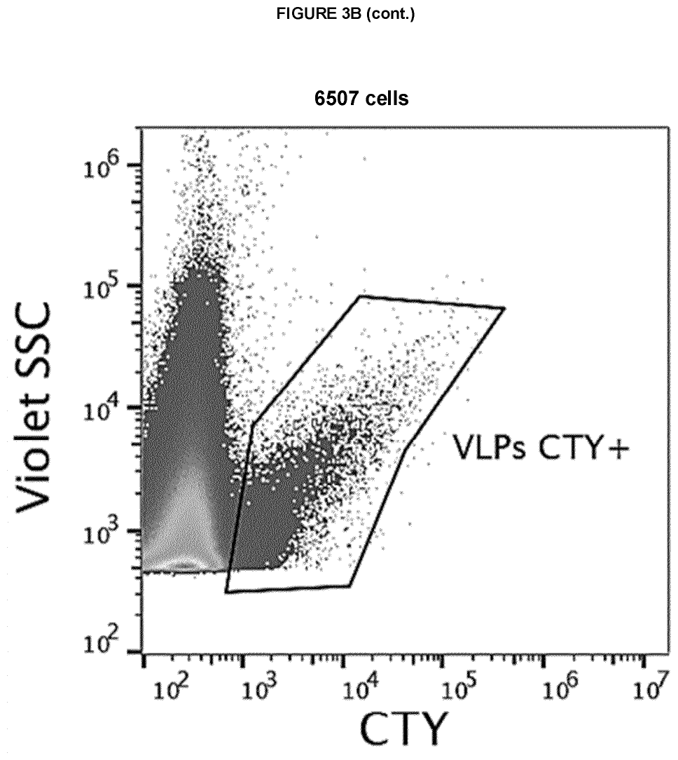

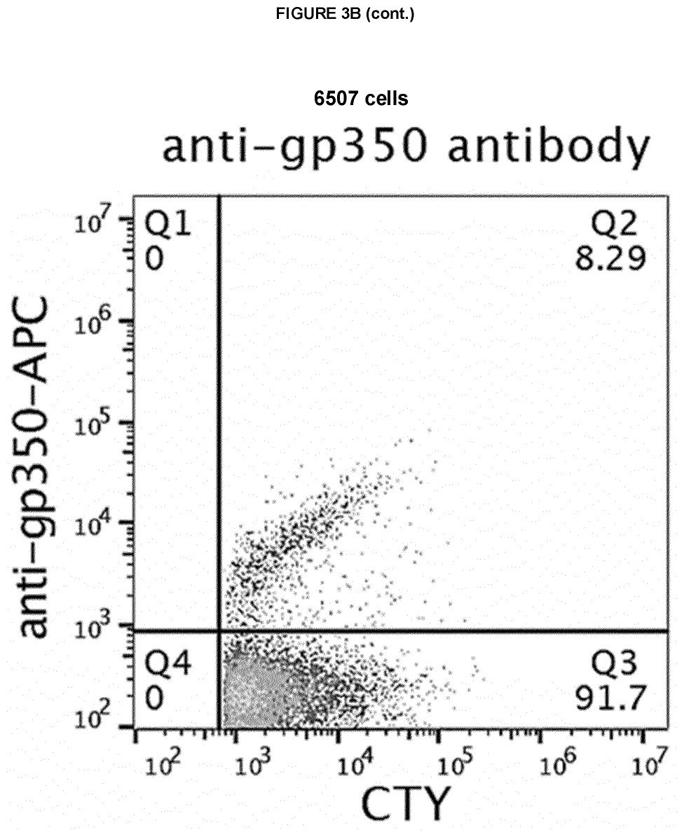

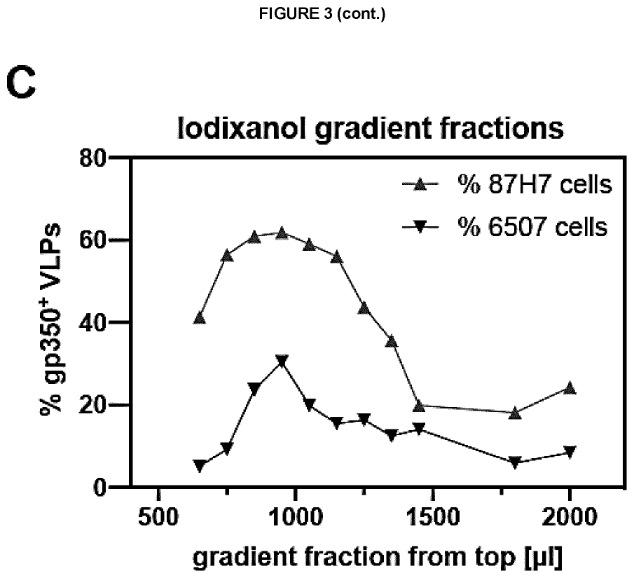

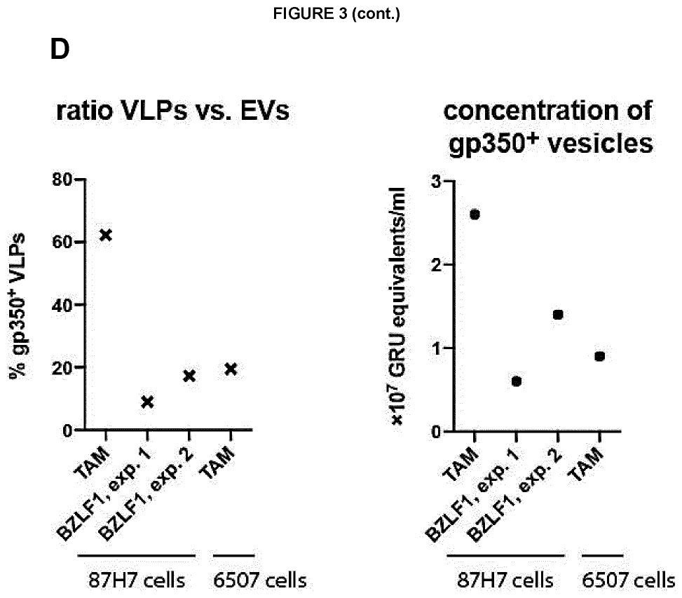

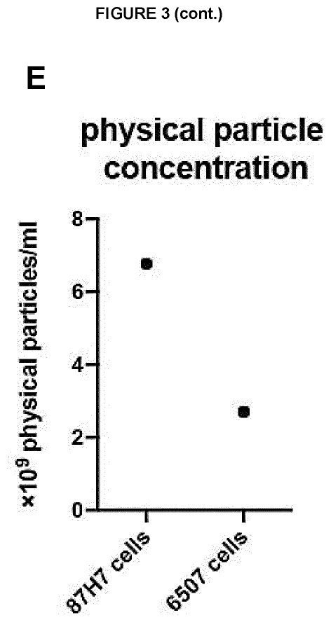

It is preferred for the composition according to the present invention that it comprises EB-VLPs and extracellular vesicles (EVs) in a ratio of 2:1 or greater, preferably of 3:1 or greater, more preferably of 5:1 or greater and even more preferably of 10:1 or greater. The ratio of EB-VLPs and extracellular vesicles (EVs) according to the present invention is determined according to the procedure as described herein in Example 3 and in FIG. 3, preferably via flow cytometry.

The present invention also provides a kit comprising EB-VLPs generated according to the method for manufacturing EB-VLPs according to the present invention.

In another aspect, the present invention also provides a method for the manufacturing of a vaccine, comprising the steps of the method for manufacturing EB-VLPs as described herein above and the further step of formulating the EB-VLPs as a vaccine.

Additionally, the present invention also provides a vaccine containing EB-VLPs obtainable by the method for manufacturing EB-VLPs as described herein.

The term “vaccine”, as employed in the present invention, means to prophylactically or therapeutically immunize an individual against EBV. The vaccine according to the present invention immunizes an individual against EBV infection and EBV-associated diseases. Immunization relates to the process of stimulating and sensitizing the immune system towards the antigen(s) within the vaccine. According to the invention, prophylactic immunization refers to the first exposure of an individual's immune system, i.e. a naïve immune system, to EBV antigens. Said first exposure results in the clearance of said antigens from the body of the exposed individual and in the development of EBV-antigen specific CD4+- and CD8+-cells and antibody-producing B-cells and long-lived memory B-cells. Upon a second exposure, the immune system is able to prevent EBV infection and/or clear said infection more effectively thereby preventing or mitigating the development of EBV-associated diseases. Specifically, the effects of said prophylactic immunization manifest itself in at least one of the following: preventing infection of the immunized individual with EBV, modifying or limiting the infection, aiding, improving, enhancing or stimulating the recovery of said individual from infection and generating immunological memory that will prevent or limit a subsequent EBV infection. The presence of any of said effects can be tested for and detected by routine methods known to the person skilled in the art. Preferably, the patient is challenged with one or more EBV antigens, which have been part of the used vaccine and antibody titers and the number of T-cells against said one or more antigens are determined. Also, the induction of neutralizing antibodies that inhibit infection of human B-cells in vitro can be determined.

In a further embodiment, the vaccine of the present invention further comprises an excipient.

The terms “carrier” and “excipient” are used interchangeably herein. Pharmaceutically acceptable carriers include, but are not limited to diluents (fillers, bulking agents, e.g. lactose, microcrystalline cellulose), disintegrants (e.g. sodium starch glycolate, croscarmellose sodium), binders (e.g. PVP, HPMC), lubricants (e.g. magnesium stearate), glidants (e.g. colloidal SiO₂), solvents/co-solvents (e.g. aqueous vehicle, propylene glycol, glycerol), buffering agents (e.g. citrate, gluconates, lactates), preservatives (e.g. Na benzoate, parabens (Me, Pr and Bu), BKC), anti-oxidants (e.g. BHT, BHA, Ascorbic acid), wetting agents (e.g. polysorbates, sorbitan esters), anti-foaming agents (e.g. Simethicone), thickening agents (e.g. methylcellulose or hydroxyethylcellulose), sweetening agents (e.g. sorbitol, saccharin, aspartame, acesulfame), flavouring agents (e.g. peppermint, lemon oils, butterscotch, etc.), humectants (e.g. propylene, glycol, glycerol, sorbitol). Further pharmaceutically acceptable carriers are (biodegradable) liposomes; microspheres made of the biodegradable polymer poly(D,L)-lactic-coglycolic acid (PLGA), albumin microspheres; synthetic polymers (soluble); nanofibers, protein-DNA complexes; protein conjugates; erythrocytes; or virosomes. Various carrier based dosage forms comprise solid lipid nanoparticles (SLNs), polymeric nanoparticles, ceramic nanoparticles, hydrogel nanoparticles, copolymerized peptide nanoparticles, nanocrystals and nanosuspensions, nanocrystals, nanotubes and nanowires, functionalized nanocarriers, nanospheres, nanocapsules, liposomes, lipid emulsions, lipid microtubules/microcylinders, lipid microbubbles, lipospheres, lipopolyplexes, inverse lipid micelles, dendrimers, ethosomes, multicomposite ultrathin capsules, aquasomes, pharmacosomes, colloidosomes, niosomes, discomes, proniosomes, microspheres, microemulsions and polymeric micelles. Other suitable pharmaceutically acceptable excipients are inter alia described in Remington's Pharmaceutical Sciences, 15th Ed., Mack Publishing Co., New Jersey (1991) and Bauer et al., Pharmazeutische Technologie, 5th Ed., Govi-Verlag Frankfurt (1997). The person skilled in the art will readily be able to choose suitable pharmaceutically acceptable carriers, depending, e.g., on the formulation and administration route of the pharmaceutical composition.

In a further embodiment, the vaccine of the present invention comprises one or more viral or non-viral polypeptides, one or more viral or non-viral nucleic acid sequences and/or vaccine adjuvants, wherein said one or more viral polypeptides or said one or more viral nucleic acid sequences are not from the same virus as the EB-VLPs.

The term “adjuvant” as used herein refers to a substance that enhances, augments or potentiates the host's immune response (antibody and/or cell-mediated) to an antigen or fragment thereof. Exemplary adjuvants for use in accordance with the present invention include inorganic compounds such as alum, aluminum hydroxide, aluminum phosphate, calcium phosphate hydroxide, the TLR9 agonist CpG oligodeoxynucleotide, the TLR4 agonist monophosphoryl lipid (MPL), the TLR4 agonist glucopyranosyl lipid (GLA), the water in oil emulsions Montanide ISA 51 and 720, mineral oils, such as paraffin oil, virosomes, bacterial products, such as killed bacteria Bordetella pertussis, Mycobacterium bovis, toxoids, nonbacterial organics, such as squalene, detergents (Quil A), and cytokines, such as IL-1, IL-2, IL-10 and IL-12. Generally, the adjuvant used in accordance with the present invention preferably potentiates the immune response to the multimeric complex of the invention and/or modulates it towards the desired immune responses.

In this specification, a number of documents including patent applications and manufacturer's manuals is cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. The term “at least one” refers, if not particularly defined differently, to one or more such as two, three, four, five, six, seven, eight, nine, ten or more. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “less than” or in turn “more than” does not include the concrete number.

For example, less than 20 mean less than the number indicated. Similarly, more than or greater than means more than or greater than the indicated number, e.g. more than 80% means more than or greater than the indicated number of 80%.

Throughout this specification and the claims which follow, unless the context requires otherwise, the word “comprise”, and variations such as “comprises” and “comprising”, will be understood to imply the inclusion of a stated integer or step or group of integers or steps, but not the exclusion of any other integer or step or group of integer or step. When used herein the term “comprising” can be substituted with the term “containing” or “including” or sometimes when used herein with the term “having”. When used herein “consisting of” excludes any element, step, or ingredient not specified.

The term “including” means “including but not limited to”. “Including” and “including but not limited to” are used interchangeably.

The term “about” means plus or minus 10%, preferably plus or minus 5%, more preferably plus or minus 2%, most preferably plus or minus 1%.

Throughout the description and claims of this specification, the singular encompasses the plural unless the context otherwise requires. In particular, where the indefinite article is used, the specification is to be understood as contemplating plurality as well as singularity, unless the context requires otherwise.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent applications, scientific publications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

The present invention is further characterized by the following items:
1. A method for manufacturing a HEK293 cell line,
   which is capable of producing Epstein-Barr virus-like particles (EB-VLPs), comprising
   (a) introducing a vector comprising the EBV genome into said cell line, said vector being capable of being propagated both in a prokaryotic and eukaryotic host cell and being capable of autonomously replicating in said cell line; and
   (b) removing from said vector nucleotide sequences required for its propagation in a prokaryotic host cell.
2. The method for manufacturing a HEK293 cell line of item 1, wherein the vector comprising the EBV genome, in comparison to a wildtype EBV genome, is modified to lack one or more sequences that are required for the packaging of said wildtype EBV genome, is modified to lack one or more sequences encoding EBV polypeptides required for said packaging, is modified to lack one or more sequences encoding EBV polypeptides required for cleavage of viral DNA prior to said packaging, and/or is modified to comprise one or more sequences encoding EBV polypeptides, whose packaging capacity is disabled.
3. The method for manufacturing a HEK293 cell line of item 1 or 2, wherein the vector comprising the EBV genome, in comparison to a wildtype EBV genome, is modified to lack one or more sequences encoding EBV polypeptides that are required for B-cell transformation and/or is modified to comprise one or more sequences encoding EBV polypeptides, whose B-cell transformation capacity is disabled.
4. The method for manufacturing a HEK293 cell line of any one of the previous items, wherein the vector comprising the EBV genome, in comparison to a wildtype EBV genome, is modified to lack one or more sequences encoding EBV polypeptides that are required for inducing replication of an EBV and/or is modified to comprise one or more sequences encoding EBV polypeptides, whose capacity for inducing EBV replication is disabled.
5. The method for manufacturing a HEK293 cell line of any one of the previous items, wherein the vector comprising the EBV genome, in comparison to a wildtype EBV genome, is modified to lack one or more expressible gene(s) selected from the group consisting of the BFLF1 gene, the BBRF1 gene, the BGRF1 gene, the BDRF1 gene, the BALF3 gene, the BFRF1A gene, and the BFRF1 gene.
6. The method for manufacturing a HEK293 cell line of any one of the previous items, wherein the vector comprising the EBV genome, in comparison to a wildtype EBV genome, is modified to lack one or more sequences encoding EBV polypeptides selected from the group consisting of the BNRF1 polypeptide, the BPLF1 polypeptide, the BGLF3 polypeptide, the BRRF2 polypeptide, the BKRF4 polypeptide and the BXLF1 polypeptide.
7. The method for manufacturing a HEK293 cell line of any one of the previous items, wherein the vector comprising the EBV genome, in comparison to a wildtype EBV genome, is modified to lack one or more miRNAs, selected from the group consisting of miR-BHRF1-1, miR-BHRF1-2, miR-BHRF1-3, miR-BART1, miR-BART2, miR-BART3, miR-BART4, miR-BART5, miR-BART6, miR-BART7, miR-BART8, miR-BART9, miR-BART10, miR-BART11, miR-BART12, miR-BART13, miR-BART14, miR-BART15, miR-BART16, miR-BART17, miR-BART18, miR-BART19, miR-BART20, miR-BART21, and miR-BART22.
8. The method for manufacturing a HEK293 cell line of item 3, wherein the one or more EBV polypeptides required for B-cell transformation, which are lacking, are selected from the group consisting of LMP-1, LMP-2, EBNA-1, EBNA-2, EBNA-LP, EBNA-3A, EBNA-3B and EBNA-3C.
9. The method for manufacturing a HEK293 cell line of item 4, wherein the one or more EBV polypeptides that are required for inducing replication of an EBV, which are lacking, or said one or more EBV polypeptides, whose capacity for inducing EBV replication is disabled, are selected from the group consisting of BZLF1, BRLF1, BMLF1 and any combination thereof.
10. The method for manufacturing a HEK293 cell line of item 4 or item 9, wherein the one or more EBV polypeptides that are required for inducing replication of an EBV, which are lacking, or said one or more EBV polypeptides, whose capacity for inducing EBV replication is disabled, is BZLF1.
11. The method for manufacturing a HEK293 cell line of any one of the previous items, wherein step b) comprises modifying the vector to be free of non-viral sequences, except for a nucleotide sequence enabling the vector to be selectioned in the cell line.
12. The method for manufacturing a HEK293 cell line of any one of the previous items, further comprising introducing an EBV gene encoding a polypeptide involved in the induction of the lytic cycle.
13. The method for manufacturing a HEK293 cell line of item 12, wherein said polypeptide involved in the induction of the lytic cycle is BZLF1, BRLF1, BMRF1, BMLF1, BALF2, BALF5, BGLF2, BHRF1, BALF4, BDLF3, or any combination thereof.
14. The method for manufacturing a HEK293 cell line of item 12 or 13, wherein said polypeptide involved in the induction of the lytic cycle is a fusion protein between BZLF1 and an estrogen receptor.
15. A HEK293 cell line obtainable by any of the methods of items 1 to 14.
16. The HEK293 cell line of item 15, wherein the vector is free of non-viral sequences, except for a nucleotide sequence enabling the vector to be selectioned in the cell line.
17. The HEK293 cell line of item 15 or item 16, comprising an EBV gene encoding a polypeptide involved in the induction of the lytic cycle, preferably wherein said polypeptide involved in the induction of the lytic cycle is BZLF1, BRLF1, BMRF1, BMLF1, BALF2, BALF5, BGLF2, BHRF1, BALF4, BDLF3, or any combination thereof or wherein said polypeptide involved in the induction of the lytic cycle is a fusion protein between BZLF1 and an estrogen receptor.
18. A method for manufacturing EB-VLPs, comprising
   (a) culturing the HEK293 cell line of any one of items 15 to 17;
   (b) inducing the lytic cycle;
   (c) obtaining EB-VLPs; and optionally
   (d) purifying said EB-VLPs.
19. The method for manufacturing EB-VLPs of item 18, wherein step (b) further comprises the addition of estrogen, tamoxifen or derivatives thereof.
20. The method for manufacturing EB-VLPs of item 18 or item 19, wherein step (d) comprises high speed centrifugation, ultracentrifugation and/or flotation density gradient centrifugation.
21. A composition comprising EB-VLPs obtainable by the method of any one of items 18 to 20.
22. The composition according to item 21, for use as a vaccine.
23. The composition according to item 21, for use in the treatment and/or prevention of a disease.
24. The composition of any one of items 21 to 23, which comprises EB-VLPs and extracellular vesicles (EVs) in a ratio of 2:1 or greater, preferably of 3:1 or greater, more preferably of 5:1 or greater and even more preferably 10:1 or greater.
25. Kit comprising EB-VLPs generated according to the method of any one of items 18 to 20.
26. A method for the manufacturing of a vaccine, comprising the steps of the method of any one of items 18 to 20 and the further step of formulating the EB-VLPs as a vaccine.
27. A vaccine containing EB-VLPs obtainable by the method according to any one of items 18 to 20.

A better understanding of the present invention and of its advantages will be gained from the following examples, offered for illustrative purposes only. The examples are not intended to limit the scope of the present invention in any way.

### EXAMPLES

Materials and Methods

Cell Lines and Cell Culture

The HEK293-based EB-VLP producer cell lines were maintained in RPMI 1640 medium (Life Technologies). All media were supplemented with 10% FBS (Life Technologies), penicillin (100 U/ml; Life Technologies), and streptomycin (100 mg/ml; Life Technologies). Cells were cultivated at 37° C. in a 5% CO 2 incubator. HEK293 cells were obtained from the Leibniz Institut Deutsche Sammlung von Mikroorganismen and Zellkulturen GmbH (DSMZ, Braunschweig, Germany). Virus-like particle producer cell lines were established after individual transfection of the EB-VLP genomic DNAs into HEK293 cells and their subsequent selection with puromycin in the range of 0.5 to 1.0 μg/ml puromycin.

Genetic Engineering of EB-VLP Genomes in E. coli

All modifications of maxi-EBV plasmids described herein were based on published techniques using homologous recombination in E. coli with linear DNA fragments (Warming et al. 2005). More advanced and recent technical steps are described in great detail in Pich et al., in the chapter “Construction of mutant EBVs” in the Section “Materials and Methods” and in “Supplemental Material” (Pich et al., 2019). For this invention, all recombinant EB-VLP genomes were based on the maxi-EBV plasmid ΔmiR (4027) (Seto et al., 2010), which comprises the entire B95-8 EBV genome cloned onto a mini-F-factor plasmid in E. coli (Delecluse et al. 1998). In contrast to the B95-8 EBV genome, ΔmiR (4027) was deficient with respect to all EBV miRNA loci and did not produce viral miRNAs as published (Seto et al., 2010).

Induction of EB-VLPs from EB-VLP Producer Cells

EB-VLP producer cells were seeded with a density of 1.1×10⁵ cells/cm² in fully complemented cell culture medium supplemented with puromycin for 24 hours. Then, the medium war replaced with RPM11640 cell culture medium, without supplements, but with 1 μM 4-hydroxy-tamoxifen. Cells were kept for 4 days, when the supernatant with the EB-VLPs was collected and processed further as needed.

### Example 1

Removal of Nucleotide Sequences Required for Propagation in a Prokaryotic Host Cell (“Removal of the Prokaryotic Backbone”) in EB-VLP Producer Genomes

First, the inventors of the present invention re-engineered the EBV genome that encodes all viral genes and it was introduced stably into HEK293 cells to establish EV-VLP producer cells. The design of the EBV genome that can support the release of VLPs is based on the well-known biology of EBV and an unlimited access to modify the genetic composition of the EBV genome in E. coli, shown in previous work of the inventors of the present invention (Delecluse et al., 1998; Delecluse et al., 1999; Pich et al., 2019).

In short, the EBV genome carried nucleotide sequences required for its propagation in a prokaryotic host cell (in the following called: “a/ the prokaryotic backbone”) derived from the mini-F′ replicon that supports the propagation of EBV genome as a single copy plasmid in E. coli cells, such as the DH10B strain and its derivatives. To maintain the EBV genome, the prokaryotic backbone was equipped with a prokaryotic gene encoding resistance against an antibiotic. In our example, the inventors used the gene chloramphenicol acetyl-transferase (SEQ ID NO: 1) and selected the E. coli cells that carry the recombinant EBV genome with chloramphenicol. The EBV genome replicated in E. coli cells, because the prokaryotic backbone contained two additional genes (ParA (SEQ ID NO: 2) and ParB (SEQ ID NO: 3)) and the mini-F′ origin of DNA replication (SEQ ID NO: 4) (see also FIG. 2A). Together, these are four genetic elements that are indispensable to propagate the EBV genome in bacteria. In E. coli cells the genetic composition of the EBV genome can be altered at will, because the viral genome acts as a (huge) DNA insert with no functional contribution to the propagation of the mini-F′ plasmid in prokaryotic cells. Genetic modifications include the deletion or functional impairment of viral genes and viral cis-acting elements that are, e.g., key to prevent the packaging of viral DNA into VLPs later in HEK293 EB-VLP producer cells (see below). These viral genes and cis-acting elements are known in the field. Additional genetic modifications encompassed the removal or inactivation of viral genes that are a risk to human health (see FIG. 2). For example, LMP1 is a viral latent gene product (SEQ ID NO: 5) that has been recognized as EBV's key oncoprotein. Thus, it was advisable to delete or inactivate LMP1's encoding gene termed BNLF1 in the EB-VLP producer cells. Additional viral genes are known immunoevasins and dampen the antigenicity of EB-VLPs (Albanese et al., 2016; Tagawa et al., 2016; Albanese et al., 2017). Consequently, all viral micro RNAs (miRNAs) were removed from the EB-VLP producer genome as described in WO 2017/148928 A1 (Means and methods for treating herpesvirus infection).

To maintain the EBV genome in HEK293 cells and to allow their handling and propagation in cell culture, the prokaryotic backbone was further equipped with at least two genes (SEQ ID NO: 7 and SEQ ID NO: 15) that were expressed from ectopic metazoan promoters in HEK293 cells. These genes encode resistance against antibiotics that are used to maintain the EB-VLP genome stably in HEK293 cells. Examples are genes that provide resistance against hygromycin (Delecluse et al., 1999) (SEQ ID NO: 7) or puromycin (Pich et al., 2019) (SEQ ID NO: 15). To monitor the presence of the EB-VLP genomic DNA in HEK293 cells (and other cells), phenotypic marker genes such as fluorescent proteins (green fluorescence protein, gfp) (SEQ ID NO: 6) were also contained in the prokaryotic backbone of the recombinant EBV genomes (Pich et al., 2019 and references therein). Together, the prokaryotic backbone was of considerable size and encompassed close to 10 kbp of foreign DNA contained in the genome of the EB-VLP producer.

Second, the inventors of the present invention found a strategy to remove the entire prokaryotic backbone of the EB-VLP producer genome, such that it can still be maintained stably in HEK293 cells, e.g. the sequences flanked by lox71 and lox66 sites were deleted upon transient expression of CRE (see FIG. 4). Why the EB-VLP genome was stably maintained in HEK293 cells, under puromycin selection, can be explained as follows: oriP (as a cis-active element, FIG. 2) together with the viral gene product EBNA1 (not shown in FIG. 2) allow (i) the extrachromosomal maintenance and (ii) the replication of the EBV plasmids together with the cellular DNA in the S-phase of the cell. The inventors discovered that the prokaryotic backbone is an obstacle to EB-VLP production, as HEK293 cells equipped with the novel EB-VLP genome released more physical particles (among them are EB-VLPs). More importantly, the quality of the composition of the particles was much improved. Quality is defined as a higher ratio of EB-VLPs to extracellular vesicles (EVs) comparing HEK293 cells with EB-VLP producer genomes that differ only in this aspect.

Construction of the EB-VLP Genome p6507.8 in E. coli

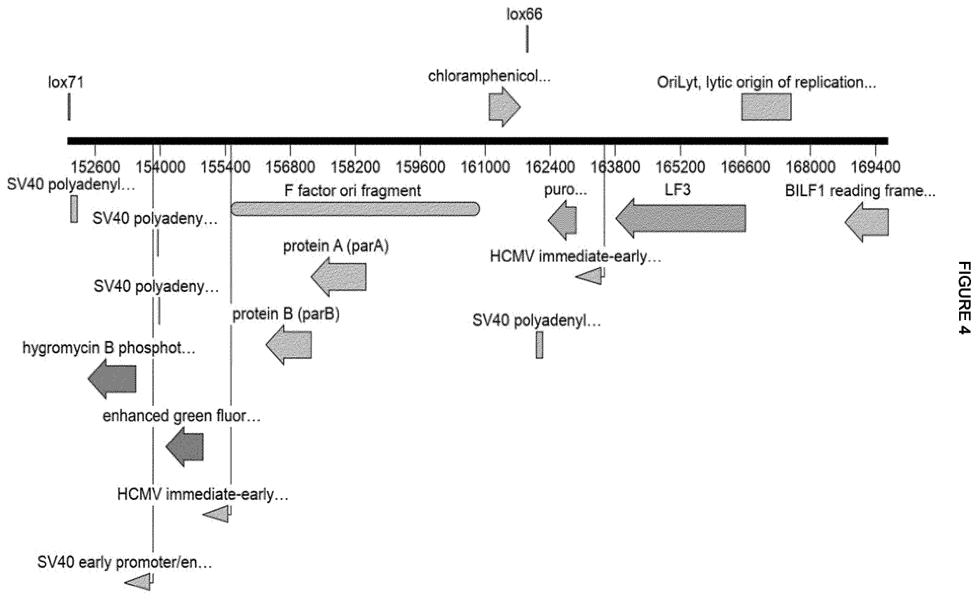

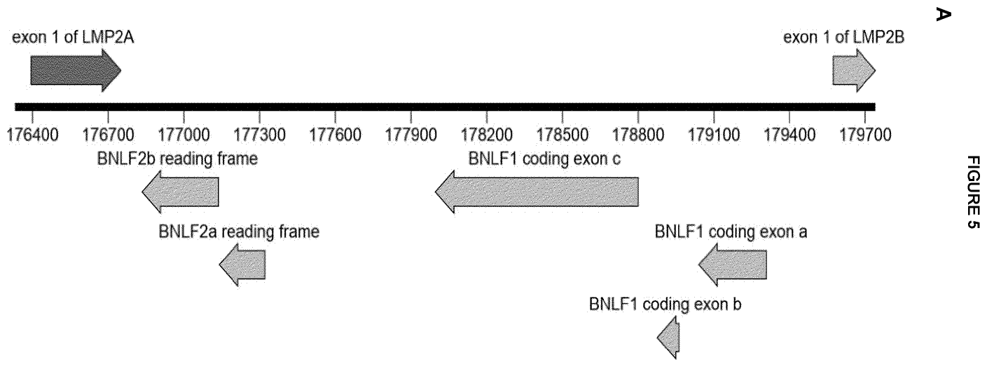

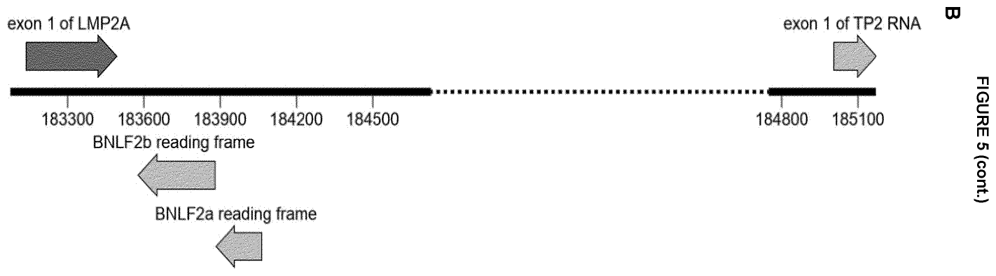

Based on an appropriate EBV genome that cannot be packaged into viral particles, because of a manipulation in one of EBV's many genes with DNA packaging functions, the inventors deleted the coding sequences of the viral oncoprotein LMP1 encoded by the viral BNFL1 gene (primary transcript given as SEQ ID NO: 8 and its three, protein-coding exon sequences given as SEQ ID NOs: 9, 10, 11) (see also FIGS. 5A and 5B), disabled the function of all viral miRNAs as described (Seto et al., 2010), and added viral sequences (SEQ ID NO: 12) that are absent in the prototypic laboratory strain B95-8 (see FIG. 4; also added was the EBV gene LF3 as shown in said figure). These additional viral sequences include, among others, the second lytic origin of DNA replication, oriLyt, similar to the EBV strain r_ΔmiR (6338) mutant EBV described in Pich et al. (Pich et al., 2019). This step also improved yield and quality of EB-VLPs produced in HEK293 cells. This EB-VLP genome was termed p6248.1.

Next, the inventors flanked the prokaryotic backbone in the p6248.1 EB-VLP genome with two modified loxP sites (SEQ ID NO: 13 and SEQ ID NO: 14) to allow the deletion of the prokaryotic backbone later in HEK293 cells. Next to one of the loxP site, the inventors also introduced a small expression cassette that can express the resistance gene for puromycin, puromycin N-acetyl-transferase (SEQ ID NO: 15), in HEK293 cells as shown in FIG. 2A to obtain the EB-VLP producer genome p6507.8 (SEQ ID NO: 20, shows nucleotide residues/coordinates #152,000-#169,676). This plasmid DNA was stably introduced in HEK293 cells using selection with puromycin and a cell clone was chosen for its good yield of EB-VLPs. This HEK293 EB-VLP producer cell clone termed 6507.43.16 served as reference and bench mark for EB-VLP production later.

Removal of the Prokaryotic Backbone in EB-VLP Producer Cells

Following this step and continuing with the engineering of the final EB-VLP producer cell line, the inventors introduced transiently an expression plasmid encoding the site-specific CRE recombinase (SEQ ID NO: 16) into this HEK293 cell clone to promote the deletion of the prokaryotic backbone in the EB-VLP producer genome contained in 6507.43.16 cells. As several copies of the EB-VLP producer genome were maintained extrachromosomally as mini-chromosomes in HEK293 cells and replicated autonomously together with the chromosomes of the cells via their latent origins of DNA replication, oriP, (see FIG. 2), all genetic elements contained within the prokaryotic backbone of the EB-VLP producer genome p6507.8 (SEQ ID NO: 20, shows nucleotide residues/coordinates #152,000-#169,676) were dispensable. To express CRE, the p6890 plasmid was introduced into 6507.43.16 cells, which were co-selected with puromycin and G418. Similar to the EB-VLP producer genome, the plasmid p6890 replicated extrachromosomally via oriP, but encoded the resistance gene neomycin phospho-transferase (SEQ ID NO: 17, shows nucleotide residues/coordinates #4824-#5618) to allow for its selection with G418. After two weeks, selection with G418 was alleviated and a variant of the parental EB-VLP producer cell line previously termed 6507.43.16 was obtained that had lost the expression of GFP indicative of the deletion of the entire prokaryotic backbone. Its deletion was confirmed by PCR and subsequent sequencing of the obtained PCR DNA products and the final cell line was termed 6507.GFPneg.5 (Cres, for short). The CRE-encoding plasmid was spontaneously lost in the proliferating cell population in a few weeks time, when selection with G418 was omitted.

### Example 2

Inducing the Release of EB-VLPs from all Producer Cells

Independent of the construction of the two EB-VLP producer cells 6507.43.16 and 6507.GFPneg.5 (Cres) described so far, the inventors developed a method to instruct all cells of EB-VLP producer cells to induce the lytic phase and release EB-VLPs into the cell culture medium.

The inventors engineered a gene (SEQ ID NO: 19) that expresses a chimeric fusion protein consisting of the open reading frame of BZLF1 fused with the hormone binding domain of the estrogen receptor. The gene was termed BZLF1:ER, which is a synthetic DNA-construct, differing from the original sequences of BZLF1 and ER. The amino acid sequence thereof is given as SEQ ID NO: 18. Technically, the synthetic gene was custom-ordered and purchased as a codon-optimized DNA fragment from a commercial supplier and molecularly cloned into a conventional retroviral vector as the only protein-encoding gene. Using standard techniques, this DNA was used to establish a retroviral vector DNA and to generate retroviral vector stocks that exclusively contain the coding information of this chimeric fusion protein. The EB-VLP producing cell lines, as engineered according to Example 1, were transduced with this vector stock to obtain cell populations, in which all cells contained this vector, together with its encoding BZLF1:ER gene (SEQ ID NO: 19) in a chromosomally integrated form and expressed from the regulatory elements of the retroviral vector backbone. As a consequence, all cells expressed the BZLF1:ER gene constitutively and at high level. See FIG. 10 for a schematic visualization of this technical step.

The constitutive expression of this gene does not harm the cells nor does the chimeric protein fusion change the biological state of the cells. The BZLF1:ER fusion protein (SEQ ID NO: 18) localizes to the cytoplasm where it is tethered to abundant so-called heat-shock proteins. This situation is reminiscent of the family of steroid hormone receptors, to which estrogen belongs. Only when the cognate hormone is present, the heat-shock bound hormone receptor dissociates from the heat-shock proteins in the cytoplasm and translocates to its site of action, the nucleus of the cell. There, it binds sequence-specifically to certain DNA binding motifs and activates the neighboring hormone-regulated genes (Pratt et al., 2004).

Similar to this mode of action, adding estrogen (or an estrogen-like hormone, such as tamoxifen or its derivatives) to the engineered EB-VLP producer cell line expressing the BZLF1:ER fusion protein releases it from heat-shock proteins in the cytoplasm. BZLF1 is a typical transcription factor that also acts as transcriptional activator in the nucleus of the cell in which the EBV genome is present in its inactive, latent form. As a transcription factor, BZLF1 encompasses several nuclear translocation signal domains, such that upon adding estrogen to the cells, the BZLF1:ER protein molecules are released from heat-shock proteins in the cytoplasm and translocate to the nucleus, where they bind the DNA genome of the EBV encoding VLPs. Thus, adding estrogen to the cells activates viral gene transcription, such that all viral lytic proteins become highly expressed, eventually. As a consequence, all cells start to assemble and release EB-VLPs into the cell culture supernatant.

### Example 3

Removal of the Prokaryotic Mini-F′ Plasmid Backbone in EB-VLP Producer Cells Leads to EB-VLPs with Superior Composition and Quality

The inventors introduced the retroviral vector encoding the BZLF1:ER fusion protein and as described in Example 2 above into the two EB-VLP producer cell lines 6507.43.16 and 6507.GFPneg.5 (Cres) that were established as described in Example 1 above. The inventors used doses of the vector that ensured the transduction of all cells in the population of cells. Having completed this step, the former cell line was termed 6507 cells, whereas the latter was termed 87H7 cells, for short.

Next, the inventors plated both cell lines at equal density and induced the production of EB-VLPs by adding tamoxifen in cell culture medium free of fetal calf serum. After four days, the inventors harvested the supernatants and processed them as described in FIG. 3A. The inventors stained the sedimented and resuspended material after pre-purification with Cell Trace Yellow (CTY) (ThermoFisher) following the manufacturer's protocol and counterstained the resuspended material with a control antibody or an antibody directed against gp350, the major glycoprotein on the surface of EBV particles. Both antibodies were conjugated with Alexa 647 to allow their detection by flow cytometry. The stained material was separated on a discontinuous iodixanol gradient by ultracentrifugation. Different fractions were harvested as illustrated in FIG. 3A following protocols as disclosed in Albanese et al. (Albanese et al., 2020).

The fractions were analyzed first by flow cytometry on a specially equipped flow cytometer (Cytoflex, Beckman Counter). Representative results are shown in FIGS. 3B and C. The data demonstrate that vesicles obtained from both cell lines (87H7 cells versus 6507 cells), typically contained in fraction 2 and 3 of iodixanol gradients, have a discrete composition when analyzed by flow cytometry. A considerable subfraction of recorded events contained vesicles that stain with CTY in all preparations. The subsequent analysis of CTY-positive vesicles revealed that vesicle preparations from supernatant from induced 87H7 cells contain up to 70% and more gp350-positive vesicles indicative of EV-VLPs. On the contrary, vesicle from induced 6507 cells contained a smaller percentage of EB-VLPs, typically around 10 to 20% (see FIG. 3B, C).

The inventors also compared the concentration of unpurified EB-VLPs directly from supernatants of the two different tamoxifen-induced cell lines using the human B cell line Elijah and a staining protocol with the same anti-gp350 antibody as in the previous paragraph. The cells were analyzed together with appropriate reference samples by flow cytometry. The inventors found that supernatants from 87H7 cells contained higher concentrations of EB-VLPs compared with supernatants from 6507 cells (see FIG. 3E).

Similarly, the inventors measured the concentration of physical particles directly in the supernatants of induced 6507 or 87H7 cells by nanotracking analysis (NTA) using a Zetaview instrument (Particle Metrix). Again, the inventors found slightly higher (two to three-fold) concentrations in supernatants from 87H7 cells compared with those from 6507 cells (see FIG. 3E).

Together, all data showed that the removal of the prokaryotic backbone in the EB-VLP genome as present in 87H7 cells supports the superior production of EB-VLPs with respect to absolute numbers and, in particular, the composition of vesicles. Clearly, the supernatants of tamoxifen-induced 87H7 cells contain a much higher ratio of gp350 positive EB-VLPs than supernatants from 6507 cells.

### Example 4

Tamoxifen Induced Production of EB-VLPs is Advantageous Compared to the Conventional Methods

The inventors also analyzed the production of EB-VLPs comparing the conventional method of inducing EB-VLP producer cells of the prior art versus the tamoxifen-induced method according to the present invention as described herein. Previously, EB-VLPs were induced in HEK293 cells by transient transfection of an expression plasmid coding for the viral transducer BZLF1 (Delecluse et al., 1999; Hettich et al., 2006).

Comparing transient transfection of the expression plasmid encoding BZLF1 versus adding the estrogen derivative tamoxifen to 87H7 EB-VLP producer cells, the inventors analyzed the composition and the numbers of EVs in cell culture supernatants from the same but differentially induced cell line.

Data shown in FIG. 3D document that the ratio of EB-VLPs versus EVs regularly exceeds 50% in supernatants of cells treated with tamoxifen, whereas the ratio in supernatants from cultured cells after transient transfection of the BZLF1 expression plasmid DNA is in the range of 10 to 20%, only. The novel approach according to the present invention and as described herein also produces higher numbers of EVs in the supernatant of the EB-VLP producer cells upon induction (see FIG. 3D).

### Conclusion

In summary, the inventors of the present invention provide herein convincing data documenting that the present invention is superior to the previous technologies in establishing, inducing and obtaining EB-VLPs from engineered HEK293 producer cells.

### REFERENCES

Albanese, M., T. Tagawa, M. Bouvet, L. Maliqi, D. Lutter, J. Hoser, M. Hastreiter, M. Hayes, B. Sugden, L. Martin, A. Moosmann, and W. Hammerschmidt. 2016. Epstein-Barr virus microRNAs reduce immune surveillance by virus-specific CD8+ T cells. Proc Natl Acad Sci USA. 113: E6467-E6475. doi: 10.1073/pnas. 1605884113.
Albanese, M., T. Tagawa, A. Buschle, and W. Hammerschmidt. 2017. microRNAs of Epstein-Barr virus control innate and adaptive anti-viral immunity. J Virol. 91:e01667-16. doi: 10.1128/JVI.01667-16.
Albanese, M., Y.-F. A. Chen, C. Huels, K. Gaertner, T. Tagawa, O. T. Keppler, C. Goebel, R. Zeidler, and W. Hammerschmidt. 2020. Micro RNAs are minor constituents of extracellular vesicles and are hardly delivered to target cells. bioRxiv. 2020.05.20.106393. doi:10.1101/2020.05.20.106393.
Baker, M. P., A. G. Eliopoulos, L. S. Young, R. J. Armitage, C. D. Gregory, and J. Gordon. 1998. Prolonged phenotypic, functional, and molecular change in group I Burkitt lymphoma cells on short-term exposure to CD40 ligand. Blood. 92:2830-2843.
Delecluse, H. J., T. Hilsendegen, D. Pich, R. Zeidler, and W. Hammerschmidt. 1998. Propagation and recovery of intact, infectious Epstein-Barr virus from prokaryotic to human cells. Proc. Natl. Acad. Sci. USA. 95:8245-8250. doi:10.1073/pnas.95.14.8245.
Delecluse, H. J., D. Pich, T. Hilsendegen, C. Baum, and W. Hammerschmidt. 1999. A first-generation packaging cell line for Epstein-Barr virus-derived vectors. Proc Natl Acad Sci USA. 96:5188-5193
Hettich, E., A. Janz, R. Zeidler, D. Pich, E. Hellebrand, B. Weissflog, A. Moosmann, and W. Hammerschmidt. 2006. Genetic design of an optimized packaging cell line for gene vectors transducing human B cells. Gene Ther. 13:844-856. doi:10.1038/sj.gt.3302714.
Klinke, O., R. Feederle, and H. J. Delecluse. 2014. Genetics of Epstein-Barr virus microRNAs. Semin Cancer Biol. 26:52-59. doi:10.1016/j.semcancer.2014.02.002.
Pich, D., P. Mrozek-Gorska, M. Bouvet, A. Sugimoto, E. Akidil, A. Grundhoff, S. Hamperl, P. D. Ling, and W. Hammerschmidt. 2019. First days in the life of naive human B lymphocytes infected with Epstein-Barr virus. MBio. 1010.1128/mBio.01723-19.
Pratt, W. B., M. D. Galigniana, Y. Morishima, and P. J. Murphy. 2004. Role of molecular chaperones in steroid receptor action. Essays Biochem. 40:41-58. doi:10.1042/bse0400041.
Seto, E., A. Moosmann, S. Gromminger, N. Walz, A. Grundhoff, and W. Hammerschmidt. 2010. Micro RNAs of Epstein-Barr virus promote cell cycle progression and prevent apoptosis of primary human B cells. PLoS Pathog. 6:e1001063. doi:10.1371/journal.ppat.1001063.
Tagawa, T., M. Albanese, M. Bouvet, A. Moosmann, J. Mautner, V. Heissmeyer, C. Zielinski, D. Lutter, J. Hoser, M. Hastreiter, M. Hayes, B. Sugden, and W. Hammerschmidt. 2016. Epstein-Barr viral miRNAs inhibit antiviral CD4+ T cell responses targeting IL-12 and peptide processing. J Exp Med. 213:2065-2080. doi:10.1084/jem.20160248.
Warming S, Costantino N, Court D L, Jenkins N A, Copeland N G. 2005. Simple and highly efficient BAC recombineering using galK selection. Nucleic Acids Res 33:e36. https://doi.org/10.1093/nar/gni035.

1. A method for manufacturing a HEK293 cell line,
which is capable of producing Epstein-Barr virus-like particles (EB-VLPs), comprising
(a) introducing a vector comprising the EBV genome into said cell line, said vector being capable of being propagated both in a prokaryotic and eukaryotic host cell and being capable of autonomously replicating in said cell line; and
(b) removing from said vector nucleotide sequences required for its propagation in a prokaryotic host cell.

2. The method for manufacturing a HEK293 cell line of claim 1, wherein the vector comprising the EBV genome, in comparison to a wildtype EBV genome, is modified to lack one or more sequences that are required for the packaging of said wildtype EBV genome, is modified to lack one or more sequences encoding EBV polypeptides required for said packaging, is modified to lack one or more sequences encoding EBV polypeptides required for cleavage of viral DNA prior to said packaging, and/or is modified to comprise one or more sequences encoding EBV polypeptides, whose packaging capacity is disabled.

3. The method for manufacturing a HEK293 cell line of claim 1 or 2, wherein the vector comprising the EBV genome, in comparison to a wildtype EBV genome, is modified to lack one or more sequences encoding EBV polypeptides that are required for B-cell transformation and/or is modified to comprise one or more sequences encoding EBV polypeptides, whose B-cell transformation capacity is disabled.

4. The method for manufacturing a HEK293 cell line of any one of the previous claims, wherein the vector comprising the EBV genome, in comparison to a wildtype EBV genome, is modified to lack one or more sequences encoding EBV polypeptides that are required for inducing replication of an EBV and/or is modified to comprise one or more sequences encoding EBV polypeptides, whose capacity for inducing EBV replication is disabled.

5. The method for manufacturing a HEK293 cell line of any one of the previous claims, wherein the vector comprising the EBV genome, in comparison to a wildtype EBV genome, is modified to lack one or more expressible gene(s) selected from the group consisting of the BFLF1 gene, the BBRF1 gene, the BGRF1 gene, the BDRF1 gene, the BALF3 gene, the BFRF1A gene, and the BFRF1 gene.

6. The method for manufacturing a HEK293 cell line of any one of the previous claims, wherein the vector comprising the EBV genome, in comparison to a wildtype EBV genome, is modified to lack one or more sequences encoding EBV polypeptides selected from the group consisting of the BNRF1 polypeptide, the BPLF1 polypeptide, the BGLF3 polypeptide, the BRRF2 polypeptide, the BKRF4 polypeptide and the BXLF1 polypeptide.

7. The method for manufacturing a HEK293 cell line of any one of the previous claims, wherein the vector comprising the EBV genome, in comparison to a wildtype EBV genome, is modified to lack one or more miRNAs, selected from the group consisting of miR-BHRF1-1, miR-BHRF1-2, miR-BHRF1-3, miR-BART1, miR-BART2, miR-BART3, miR-BART4, miR-BART5, miR-BART6, miR-BART7, miR-BART8, miR-BART9, miR-BART10, miR-BART11, miR-BART12, miR-BART13, miR-BART14, miR-BART15, miR-BART16, miR-BART17, miR-BART18, miR-BART19, miR-BART20, miR-BART21, and miR-BART22.

8. The method for manufacturing a HEK293 cell line of claim 3, wherein the one or more EBV polypeptides required for B-cell transformation, which are lacking, are selected from the group consisting of LMP-1, LMP-2, EBNA-1, EBNA-2, EBNA-LP, EBNA-3A, EBNA-3B and EBNA-3C.

9. The method for manufacturing a HEK293 cell line of claim 4, wherein the one or more EBV polypeptides that are required for inducing replication of an EBV, which are lacking, or said one or more EBV polypeptides, whose capacity for inducing EBV replication is disabled, are selected from the group consisting of BZLF1, BRLF1, BMLF1 and any combination thereof.

10. The method for manufacturing a HEK293 cell line of claim 4 or claim 9, wherein the one or more EBV polypeptides that are required for inducing replication of an EBV, which are lacking, or said one or more EBV polypeptides, whose capacity for inducing EBV replication is disabled, is BZLF1.

11. The method for manufacturing a HEK293 cell line of any one of the previous claims, wherein step b) comprises modifying the vector to be free of non-viral sequences, except for a nucleotide sequence enabling the vector to be selectioned in the cell line.

12. The method for manufacturing a HEK293 cell line of any one of the previous claims, further comprising introducing an EBV gene encoding a polypeptide involved in the induction of the lytic cycle.

13. The method for manufacturing a HEK293 cell line of claim 12, wherein said polypeptide involved in the induction of the lytic cycle is BZLF1, BRLF1, BMRF1, BMLF1, BALF2, BALF5, BGLF2, BHRF1, BALF4, BDLF3, or any combination thereof.

14. The method for manufacturing a HEK293 cell line of claim 12 or 13, wherein said polypeptide involved in the induction of the lytic cycle is a fusion protein between BZLF1 and an estrogen receptor.

15. A HEK293 cell line obtainable by any of the methods of claims 1 to 14.

16. The HEK293 cell line of claim 15, wherein the vector is free of non-viral sequences, except for a nucleotide sequence enabling the vector to be selectioned in the cell line.

17. The HEK293 cell line of claim 15 or claim 16, comprising an EBV gene encoding a polypeptide involved in the induction of the lytic cycle, preferably wherein said polypeptide involved in the induction of the lytic cycle is BZLF1, BRLF1, BMRF1, BMLF1, BALF2, BALF5, BGLF2, BHRF1, BALF4, BDLF3, or any combination thereof or wherein said polypeptide involved in the induction of the lytic cycle is a fusion protein between BZLF1 and an estrogen receptor.

18. A method for manufacturing EB-VLPs, comprising
(a) culturing the HEK293 cell line of any one of claims 15 to 17;
(b) inducing the lytic cycle;
(c) obtaining EB-VLPs; and optionally
(d) purifying said EB-VLPs.

19. The method for manufacturing EB-VLPs of claim 18, wherein step (b) further comprises the addition of estrogen, tamoxifen or derivatives thereof.

20. The method for manufacturing EB-VLPs of claim 18 or claim 19, wherein step (d) comprises high speed centrifugation, ultracentrifugation and/or flotation density gradient centrifugation.

21. A composition comprising EB-VLPs obtainable by the method of any one of claims 18 to 20.

22. The composition according to claim 21, for use as a vaccine.

23. The composition according to claim 21, for use in the treatment and/or prevention of a disease.

24. The composition of any one of claims 21 to 23, which comprises EB-VLPs and extracellular vesicles (EVs) in a ratio of 2:1 or greater, preferably of 3:1 or greater, more preferably of 5:1 or greater and even more preferably 10:1 or greater.

25. Kit comprising EB-VLPs generated according to the method of any one of claims 18 to 20.

26. A method for the manufacturing of a vaccine, comprising the steps of the method of any one of claims 18 to 20 and the further step of formulating the EB-VLPs as a vaccine.

27. A vaccine containing EB-VLPs obtainable by the method according to any one of claims 18 to 20.


SEQUENCE LISTING (22 sequences)

SEQ ID NO: 1 - DNA, 660 bp, Escherichia coli
atggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaa

SEQ ID NO: 2 - DNA, 1176 bp, Escherichia coli
atgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataa

SEQ ID NO: 3 - DNA, 972 bp, Escherichia coli
atgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctga

SEQ ID NO: 4 - DNA, 5352 bp, Escherichia coli
aacgtgccggcacggcctgggtaaccaggtattttgtccacataaccgtgcgcaaaatgttgtggataagcaggacacagcagcaatccacagcaggcatacaaccgcacaccgaggttactccgttctacaggttacgacgacatgtcaatacttgcccttgacaggcattgatggaatcgtagtctcacgctgatagtctgatcgacaatacaagtgggaccgtggtcccagaccgataatcagaccgacaacacgagtgggatcgtggtcccagactaataatcagaccgacgatacgagtgggaccgtggtcccagactaataatcagaccgacgatacgagtgggaccgtggttccagactaataatcagaccgacgatacgagtgggaccgtggtcccagactaataatcagaccgacgatacgagtgggaccatggtcccagactaataatcagaccgacgatacgagtgggaccgtggtcccagtctgattatcagaccgacgatacgagtgggaccgtggtcccagactaataatcagaccgacgatacgagtgggaccgtggtcccagactaataatcagaccgacgatacgagtgggaccgtggtcccagtctgattatcagaccgacgatacaagtggaacagtgggcccagagagaatattcaggccagttatgctttctggcctgtaacaaaggacattaagtaaagacagataaacgtagactaaaacgtggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaaactagttcttgatgcagatgacgttttaagcacagaagttaaaagagtgataacttcttcagcttcaaatatcaccccagcttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattactattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcgtttttttattaaatcttgcaatttactgcaaagcaacaacaaaatcgcaaagtcatcaaaaaaccgcaaagttgtttaaaataagagcaacactacaaaaggagataagaagagcacatacctcagtcacttattatcactagcgctcgccgcagccgtgtaaccgagcatagcgagcgaactggcgaggaagcaaagaagaactgttctgtcagatagctcttacgctcagcgcaagaagaaatatccaccgtgggaaaaactccaggtagaggtacacacgcggatagccaattcagagtaataaactgtgataatcaaccctcatcaatgatgacgaactaacccccgatatcaggtcacatgacgaagggaaagagaaggaaatcaactgtgacaaactgccctcaaatttggcttccttaaaaattacagttcaaaaagtatgagaaaatccatgcaggctgaaggaaacagcaaaactgtgacaaattaccctcagtaggtcagaacaaatgtgacgaaccaccctcaaatctgtgacagataaccctcagactatcctgtcgtcatggaagtgatatcgcggaaggaaaatacgatatgagtcgtctggcggcctttctttttctcaatgtatgagaggcgcattggagttctgctgttgatctcattaacacagacctgcaggaagcggcggcggaagtcaggcatacgctggtaactttgaggcagctggtaacgctctatgatccagtcgattttcagagagacgatgcctgagccatccggcttacgatactgacacagggattcgtataaacgcatggcatacggattggtgatttcttttgtttcactaagccgaaactgcgtaaaccggttctgtaacccgataaagaagggaatgagatatgggttgatatgtacactgtaaagccctctggatggactgtgcgcacgtttgataaaccaaggaaaagattcatagcctttttcatcgccggcatcctcttcagggcgataaaaaaccacttccttccccgcgaaactcttcaatgcctgccgtatatccttactggcttccgcagaggtcaatccgaatatttcagcatatttagcaacatggatctcgcagataccgtcatgttcctgtagggtgccatcagattttctgatctggtcaacgaacagatacagcatacgtttttgatcccgggagagactatatgccgcctcagtgaggtcgtttgactggacgattcgcgggctatttttacgtttcttgtgattgataaccgctgtttccgccatgacagatccatgtgaagtgtgacaagtttttagattgtcacactaaataaaaaagagtcaataagcagggataactttgtgaaaaaacagcttcttctgagggcaatttgtcacagggttaagggcaatttgtcacagacaggactgtcatttgagggtgatttgtcacactgaaagggcaatttgtcacaacaccttctctagaaccagcatggataaaggcctacaaggcgctctaaaaaagaagatctaaaaactataaaaaaaataattataaaaatatccccgtggataagtggataaccccaagggaagttttttcaggcatcgtgtgtaagcagaatatataagtgctgttccctggtgcttcctcgctcactcgagggcttcgccgtcgctcgactgcggcgagcctactggctgtaaaaggacagaccacatcatggttctgtgttcattaggttgttctgtccattgctgacataatccgctccacttcaacgtaacaccgcacgaagatttctattgttcctgaaggcatattcaaatcgttttcgttaccgcttgcaggcatcatgacagaacactacttcctataaacgctacacaggctcctgagattaataatgcggatctctacgataatgggagattttcccgactgtttcgttcgcttctcagtggataacagccagcttctctgtttaacagacaaaaacagcatatccactcagttccacatttccatataaaggccaaggcatttattctcaggataattgtttcagcatcgcaaccgcatcagactccggcatcgcaaactgcacccggtgccgggcagccacatccagcgcaaaaaccttcgtgtagacttccgttgaactgatggacttatgtcccatcaggctttgcagaactttcagcggtataccggcatacagcatgtgcatcgcataggaatggcggaacgtatgtggtgtgaccggaacagagaacgtcacaccgtcagcagcagcggcggcaaccgcctccccaatccaggtcctgaccgttctgtccgtcacttcccagatccgcgctttctctgtccttcctgtgcgacggttacgccgctccatg

SEQ ID NO: 5 - protein, 386 aa, artificial sequence
  Protein sequence of LMP1 of Epstein-Barr virus
MEHDLERGPPGPRRPPRGPPLSSSLGLALLLLLLALLFWLYIVMSDWTGGALLVLYSFALMLIIIILIIFIFRRDLLCPLGALCILLLMITLLLIALWNLHGQALFLGIVLFIFGCLLVLGIWIYLLEMLWRLGATIWQLLAFFLAFFLDLILLIIALYLQQNWWTLLVDLLWLLLFLAILIWMYYHGQRHSDEHHHDDSLPHPQQATDDSGHESDSNSNEGRHHLLVSGAGDGPPLCSQNLGAPGGGPDNGPQDPDNTDDNGPQDPDNTDDNGPHDPLPQDPDNTDDNGPQDPDNTDDNGPHDPLPHSPSDSAGNDGGPPQLTEEVENKGGDQGPPLMTDGGGGHSHDSGHGGGDPHLPTLLLGSSGSGGDDDDPHGPVQLSYYD

SEQ ID NO: 6 - DNA, 798 bp, artificial sequence
  Gene sequence of enhanced green fluorescence protein (eGFP) of Aequorea victoria
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccgcgggcccgggatccaccggatctagataa

SEQ ID NO: 7 - DNA, 1026 bp, Escherichia coli
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatag

SEQ ID NO: 8 - DNA, 2597 bp, artificial sequence
  gene sequence of BNLF1 of Epstein-Barr virus
tacataagcctctcacactgctctgcccccttctttcctcaactgccttgctcctgacacactgccctgaggatggaacacgaccttgagaggggcccaccgggcccgcgacggccccctcgaggaccccccctctcctcttccctaggccttgctctccttctcctcctcttggcgctactgttttggctgtacatcgttatgagtgactggactggaggagccctccttgtcctctattcctttgctctcatgcttataattataattttgatcatctttatcttcagaagagaccttctctgtccacttggagccctttgtatactcctactgatgagtaagtattacaccctttgccccacaccccctttcccttactcttccttctctaacgcactttctcctctttccccagtcaccctcctgctcatcgctctctggaatttgcacggacaggcattgttccttggaattgtgctgttcatcttcgggtgcttacttggtaagatctaacattccctaggaattatttaccacacccccacttttccaaccctaacactcttttttcaacgcagtcttaggtatctggatctacttattggagatgctctggcgacttggtgccaccatctggcagcttttggccttcttcctagccttcttcctagacctcatcctgctcattattgctctctatctacaacaaaactggtggactctattggttgatctcctttggctcctcctgtttctggcgattttaatctggatgtattaccatggacaacgacacagtgatgaacaccaccacgatgactccctcccgcaccctcaacaagctaccgatgattctggccatgaatctgactctaactccaacgagggcagacaccacctgctcgtgagtggagccggcgacggacccccactctgctctcaaaacctaggcgcacctggaggtggtcctgacaatggcccacaggaccctgacaacactgatgacaatggcccacaggaccctgacaacactgatgacaatggcccacatgacccgctgcctcaggaccctgacaacactgatgacaatggcccacaggaccctgacaacactgatgacaatggcccacatgacccgctgcctcatagccctagcgactctgctggaaatgatggaggccctccacaattgacggaagaggttgaaaacaaaggaggtgaccagggcccgcctttgatgacagacggaggcggcggtcatagtcatgattccggccatggcggcggtgatccacaccttcctacgctgcttttgggttcttctggttccggtggagatgatgacgacccccacggcccagttcagctaagctactatgactaacctttctttacttctaggcattaccatgtcataggcttgcctgactgactctccctccatttactgggaatgccttagctaatcaccttaactggcacacactcccttagccacactgtctgtctaggctgaaaagccacattcatattctatttcaaaacaaggggaaaggaggacatgcgagaattggcagacacctttacccagcccttaacacaccacacaggtagcaaggacccgggcgttgccagactccgccaccaacgcccctgcgttgaacccacccctcctacacacatcagacctctgcacaacacaactaccaggcagatgaggccccttacttccacagggtactggcataccagcgggggaccacatacatccctgtctcccacccagtaactccagcaactttgctttccatcttgtgccaatacacatttggattcagcccaagccacacctaactcatgccagcagaggcaggaacacctgttgttgacacattctttgcgcataagcactttaatccctctctcacacccagaaactaagagctagcccaaaacctccacacctgtcctcgctcatctttccacattcctctggccttctttccttgtccttactgtataaaagtccacgaaaacagctgtgcctcactctcgagatggtacacgtcctggagcgtgctttgctagagcagcagtcctctgcctgcggcctgcccggctcttctacggagaccaggcctagccacccctgccccgaggacccagacgtcagcagactaagactactcctggtggtactctgtgtcctgtttggacttttatgcctgctcctcatctaagaagccaccatgcgaccgggtagaccactggctggattctacgctactctccgccgttccttcagaagaatgtccaaaaggtcaaagaacaaggccaagaaggagcgtgtccccgtggaggaccgcccaccgactccgatgcccaccagccagcgactgatccgcagaaacgcgttgggaggaggcgtccgccccgatgcggaggactgcatccaacgcttccaccccctggagccagcgctgggggtgtcaacaaagaactttgacctgttgtccctgagatgtgaattgggatggtgtggataacatctcccgctagatggcgcccttattattgatgtgacttgtgatgca

SEQ ID NO: 9 - DNA, 268 bp, artificial sequence
  Exon a of gene sequence of BNLF1 of Epstein- Barr virus
atggaacacgaccttgagaggggcccaccgggcccgcgacggccccctcgaggaccccccctctcctcttccctaggccttgctctccttctcctcctcttggcgctactgttttggctgtacatcgttatgagtgactggactggaggagccctccttgtcctctattcctttgctctcatgcttataattataattttgatcatctttatcttcagaagagaccttctctgtccacttggagccctttgtatactcctactgatga

SEQ ID NO: 10 - DNA, 87 bp, artificial sequence
  Exon b of gene sequence of Epstein-Barr virus
tcaccctcctgctcatcgctctctggaatttgcacggacaggcattgttccttggaattgtgctgttcatcttcgggtgcttacttg

SEQ ID NO: 11 - DNA, 803 bp, artificial sequence
  Exon c of gene sequence of BNLF1 of Epstein- Barr virus
tcttaggtatctggatctacttattggagatgctctggcgacttggtgccaccatctggcagcttttggccttcttcctagccttcttcctagacctcatcctgctcattattgctctctatctacaacaaaactggtggactctattggttgatctcctttggctcctcctgtttctggcgattttaatctggatgtattaccatggacaacgacacagtgatgaacaccaccacgatgactccctcccgcaccctcaacaagctaccgatgattctggccatgaatctgactctaactccaacgagggcagacaccacctgctcgtgagtggagccggcgacggacccccactctgctctcaaaacctaggcgcacctggaggtggtcctgacaatggcccacaggaccctgacaacactgatgacaatggcccacaggaccctgacaacactgatgacaatggcccacatgacccgctgcctcaggaccctgacaacactgatgacaatggcccacaggaccctgacaacactgatgacaatggcccacatgacccgctgcctcatagccctagcgactctgctggaaatgatggaggccctccacaattgacggaagaggttgaaaacaaaggaggtgaccagggcccgcctttgatgacagacggaggcggcggtcatagtcatgattccggccatggcggcggtgatccacaccttcctacgctgcttttgggttcttctggttccggtggagatgatgacgacccccacggcccagttcagctaagctactatgac

SEQ ID NO: 12 - DNA, 5019 bp, artificial sequence
  viral sequence of Epstein-Barr virus
ctttcctcacaatacaaatgttactaacgtctgccctctggagacctgctatgtggctagacgtatggcctacccaagacgttgggggtctcgggtaggccatgattcttccaggcataggttacaaccagtcactgctatcaagcctactcagttcccaacgcagcacataccccccgcctctcctgccatgaggacttatggcagtgtttactgttctgcttttactcttggaccaggctgtcattctatcagaataacaggggaagcaaggccccctgcttcagcgggacacgtgtttctagaatctcggagccaataactacctgcccctctaatctgtatgctgcatgaaaaaccacatacacgtgatgtaagtttagccagtttattgttacaccaatgccccgaaagtctccccctgtccctttgggtctcaggacccagccctggagctcgggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggcccgctgccccgctccggcggggggtggccggctgcagccgggtccggggttccggccctggagctcggggggcggccgggtggcccaccgggtccgctgggtccgctgccccgctccggcgggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcgggggacggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgagtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggcccgctgccccgctccggcggggatgggggtgcgctcccaggccggaccctggtgccaggcagggaccccgcgccacccgcttcatggggggggaggccgccgcaaggacgccgggccggctgggaggtgtgcaccccccgagcgtctggacgacgctggcgagccgggccagctcgccttcttttatcctctttttggggtttctgtgcaataccttaaggtttgctcaggtgtggggggcttctcattggttaattcaggtgtgtgattttagcccgttgggttacattaaggtgtgtaaccaggtgggtggtacctggaggtcattctattgggataacgagaggaggaggggctagaggcccgcgagatttggggtaggcggagcctcaggagggtcccctccatagggttgaaccaggagggggaggagtgggctccgccccgatatacctagtgggtggagcctagaggtaggtctccatagggttccattatcctggaggtatcctaagctccgcccctatataccaggtgggtggagctaggtaggattcagctaggttcctactggggtacccccctaccctaccttaaggtgcgccacccttcctccttccgttttaatggtagaataacctataggttattaacctagtggtggaatagggtattgcagctgggtatatacctataggtatatagaacctagaggaagggaaccctatagtgtaatccctcccccccctacccccccctcccttacggttgcctgagcccatcccccaccccagcaccccggggtgacgtggccccccgcgtgccttactgacttgtcacctttgcacatttggtcagctgaccgatgctcgccacttcctgggtcatgacctggcctgtgccttgtcccgtggacaatgtccctccagcgtggtggctgcctttgggatgcatcactttgagccactaagcccccgttgctcgccttgcctgcctcaccatgacacactaagcccctgctaatccatgagccccgcctttaggaagcaccacgtcccggggacggaagctggattttggccagtcttcaatttttgggagtggttttgtgtgagccggaagttggcaatggggtgagggcggcgctggttaagctgacgacctcccaaggtctctcaccctgggtacacaggtggggcggcagcctctaactttggctgtggcctctatttcctccctttcctagccagggccatgtgttcctgcatgtctacttgcctcctgtggtggcagagcttggccctgtgcccaacccccgccttgggagcctgtaggggccaacacccttggtttgtttgtgttcctgtttgctggcaacttactggcagccgagcagattctaatgggcgcccgccttctttctctcttgttttattaatagaatctcagccaggacctatacctgagacttcaaagtctggtcctgggttctgagacccccaagatttgtcatgcacacctgcacacctgttggtattgggtttctattcttgagtgtgaaagtttgtaaaaaaattcataaaatgtcactaattcctcttacctgtttagggtattgtgcaattcttcagcctgcctattttcaatttgcctaaggtggcaatttaagatgtggttaattaaccattttcctgtctgacaccactgcatgggcaaccgggttccatggcacatttagagataaacatagatgtcttgtcttgctcatgtgcagaggagggggtgttggtgtgcaatatagtttctggattccaaattgagttgggggtgctattttcactatggaattaaattactgacattagacagtggacacccggctatatgtggggatgtctgtggcttgtcatttcctcttagaaggtaatcccccatcttaacttccctttaaattgtgatgcaagccctgggttatttatagaatgattatctaggtttgatagtctgaaggctgggcagagaatgtttgtaatttttattcaccttctttaccccccacgagtatccagttctagaa

SEQ ID NO: 13 - DNA, 34 bp, artificial sequence
  sequence of modified loxP site
taccgttcgtatagggtaggctatacgaagttat

SEQ ID NO: 14 - DNA, 34 bp, artificial sequence
  sequence of modified loxP site
ataacttcgtatagggtaggctatacgaacggta

SEQ ID NO: 15 - DNA, 600 bp, artificial sequence
  gene sequence of puromycin N-acetyl transferase (pac) of Streptomyces alboniger
atgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctaa

SEQ ID NO: 16 - DNA, 1053 bp, artificial sequence
  gene sequence for encoding site-specific CRE recombinase of P1 bacteriophage
atgcccaagaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattag

SEQ ID NO: 17 - DNA, 795 bp, artificial sequence
  gene sequence of resistance gene neomycin transferase (neo) of bacterial transposon Tn5
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 18 - protein, 567 aa, artificial sequence
  fusion protein BZLF1ER
MMDPNSTSEDVKFTPDPYQVPFVQAFDQATRVYQDLGGPSQAPLPCVLWPVLPEPLPQGQLTAYHVSTAPTGSWFSVPQPAPENAYQAYAAPQLFPVSDITQNQQTNQAGGEAPQPGDNSTVQTAAAVVFACPGANQGQQLADIGVPQPAPVAAPARRTRKPQQPESLEECDSELEIKRYKNRVASRKCRAKFKQLLQHYREVAAAKSSENDRLRLLLKQMCPSLDVDSIIPRTPDVLHEDLLNFSDPRNEMGASGDMRAANLWPSPLVIKHTKKNSPALSLTADQMVSALLDAEPPMIYSEYDPSRPFSEASMMGLLTNLADRELVHMINWAKRVPGFGDLNLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHRRLAQLLLILSHIRHMSNKRMEHLYNMKCKNVVPLYDLLLEMLDAHRLHAPASRMGVPPEEPSQTQLATTSSTSAHSLQTYYIPPEAEGFPNTI

SEQ ID NO: 19 - DNA, 1704 bp, artificial sequence
  gene sequence of BZLF1ER
atgatggacccaaactcgacttctgaagatgtaaaatttacacctgacccataccaggtgccttttgtacaagcttttgaccaagctaccagagtctatcaggacctgggagggccatcgcaagctcctttgccttgtgtgctgtggccggtgctgccagagcctctgccacaaggccagctaactgcctatcatgtttcaaccgctccgactgggtcgtggttttctgtccctcagcctgctcctgagaatgcttatcaagcttatgcagcacctcagctgttcccagtctccgacataacccagaatcaacagactaaccaagccgggggagaagcacctcaacctggagacaattctactgttcaaacagcagcagcagtggtgtttgcttgccccggggctaaccaaggacaacagctagcagacattggtgttccacagcctgcaccagtggctgccccggcacgacgcacacggaaaccacaacagccagaatcgctggaggaatgcgattctgaactagaaataaagcgatacaagaatcgggtggcttccagaaaatgccgggccaagtttaagcaactgctgcagcactaccgtgaggtggctgctgccaaatcatctgaaaatgacaggctgcgcctcctgttgaagcagatgtgcccaagcctggatgttgactccattatcccccggacaccagatgttttacacgaggatctcttaaatttctcggatccacgaaatgaaatgggtgcttcaggagacatgagggctgccaacctttggccaagccctcttgtgattaagcacactaagaagaatagccctgccttgtccttgacagctgaccagatggtcagtgccttgttggatgctgaaccgcccatgatctattctgaatatgatccttctagacccttcagtgaagcctcaatgatgggcttattgaccaacctagcagatagggagctggttcatatgatcaactgggcaaagagagtgccaggctttggggacttgaatctccatgatcaggtccaccttctcgagtgtgcctggctggagattctgatgattggtctcgtctggcgctccatggaacacccggggaagctcctgtttgctcctaacttgctcctggacaggaatcaaggtaaatgtgtggaaggcatggtggagatctttgacatgttgcttgctacgtcaagtcggttccgcatgatgaacctgcagggtgaagagtttgtgtgcctcaaatccatcattttgcttaattccggagtgtacacgtttctgtccagcaccttgaagtctctggaagagaaggaccacatccaccgtgtcctggacaagatcacagacactttgatccacctgatggccaaagctggcctgactctgcagcagcagcatcgccgcctagctcagctccttctcattctttcccatatccggcatatgagtaacaaacgcatggagcatctctacaacatgaaatgcaagaacgtggtacccctctatgacctgctcctggagatgttggatgcccaccgccttcatgccccagccagtcgcatgggagtgcccccagaggagcccagccagacccagctggccaccaccagctccacttcagcacattccttacaaacctactacatacccccggaagcagagggcttccccaacacgatctga

SEQ ID NO: 20 - DNA, 17677 bp, artificial sequence
  nucleotide sequence of p6507.8
aaaccggtgggccagggccttaccgttcgtatagggtaggctatacgaagttatggccgcacgggccatcgatgcggatccagacatgataagatacattgatgagtttggacaaaccacaactagaatgcagtgaaaaaaatgctttatttgtgaaatttgtgatgctattgctttatttgtaaccattataagctgcaataaacaagttgcccgggcccgagtgcgtgggcatgacaatcgtgccctggggaccaacacaatccagaagggcctgaatcactgcgaccggccctcccgcgacccagccgagcgagcttagcgaactgtggacgagaactgtgccaccaagcgtaaggccgttctctcgcatttgccttgctaggctcgcgcgagttgctggctgaggcgttctcgaaatcagctcttgttcggtcggcatctactctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatggtggagggcccccccgcagatctgagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagccaagcttctcgacagcgacacacttgcatcggatgcagcccggttcgcgttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcagttatctagatccggtggatcccgggcccgcggtaccgtcgactgcagaattcgaagcttgagctcgagatctgagtccggacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtagcgctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaaacgtgccggcacggcctgggtaaccaggtattttgtccacataaccgtgcgcaaaatgttgtggataagcaggacacagcagcaatccacagcaggcatacaaccgcacaccgaggttactccgttctacaggttacgacgacatgtcaatacttgcccttgacaggcattgatggaatcgtagtctcacgctgatagtctgatcgacaatacaagtgggaccgtggtcccagaccgataatcagaccgacaacacgagtgggatcgtggtcccagactaataatcagaccgacgatacgagtgggaccgtggtcccagactaataatcagaccgacgatacgagtgggaccgtggttccagactaataatcagaccgacgatacgagtgggaccgtggtcccagactaataatcagaccgacgatacgagtgggaccatggtcccagactaataatcagaccgacgatacgagtgggaccgtggtcccagtctgattatcagaccgacgatacgagtgggaccgtggtcccagactaataatcagaccgacgatacgagtgggaccgtggtcccagactaataatcagaccgacgatacgagtgggaccgtggtcccagtctgattatcagaccgacgatacaagtggaacagtgggcccagagagaatattcaggccagttatgctttctggcctgtaacaaaggacattaagtaaagacagataaacgtagactaaaacgtggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaaactagttcttgatgcagatgacgttttaagcacagaagttaaaagagtgataacttcttcagcttcaaatatcaccccagcttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattactattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcgtttttttattaaatcttgcaatttactgcaaagcaacaacaaaatcgcaaagtcatcaaaaaaccgcaaagttgtttaaaataagagcaacactacaaaaggagataagaagagcacatacctcagtcacttattatcactagcgctcgccgcagccgtgtaaccgagcatagcgagcgaactggcgaggaagcaaagaagaactgttctgtcagatagctcttacgctcagcgcaagaagaaatatccaccgtgggaaaaactccaggtagaggtacacacgcggatagccaattcagagtaataaactgtgataatcaaccctcatcaatgatgacgaactaacccccgatatcaggtcacatgacgaagggaaagagaaggaaatcaactgtgacaaactgccctcaaatttggcttccttaaaaattacagttcaaaaagtatgagaaaatccatgcaggctgaaggaaacagcaaaactgtgacaaattaccctcagtaggtcagaacaaatgtgacgaaccaccctcaaatctgtgacagataaccctcagactatcctgtcgtcatggaagtgatatcgcggaaggaaaatacgatatgagtcgtctggcggcctttctttttctcaatgtatgagaggcgcattggagttctgctgttgatctcattaacacagacctgcaggaagcggcggcggaagtcaggcatacgctggtaactttgaggcagctggtaacgctctatgatccagtcgattttcagagagacgatgcctgagccatccggcttacgatactgacacagggattcgtataaacgcatggcatacggattggtgatttcttttgtttcactaagccgaaactgcgtaaaccggttctgtaacccgataaagaagggaatgagatatgggttgatatgtacactgtaaagccctctggatggactgtgcgcacgtttgataaaccaaggaaaagattcatagcctttttcatcgccggcatcctcttcagggcgataaaaaaccacttccttccccgcgaaactcttcaatgcctgccgtatatccttactggcttccgcagaggtcaatccgaatatttcagcatatttagcaacatggatctcgcagataccgtcatgttcctgtagggtgccatcagattttctgatctggtcaacgaacagatacagcatacgtttttgatcccgggagagactatatgccgcctcagtgaggtcgtttgactggacgattcgcgggctatttttacgtttcttgtgattgataaccgctgtttccgccatgacagatccatgtgaagtgtgacaagtttttagattgtcacactaaataaaaaagagtcaataagcagggataactttgtgaaaaaacagcttcttctgagggcaatttgtcacagggttaagggcaatttgtcacagacaggactgtcatttgagggtgatttgtcacactgaaagggcaatttgtcacaacaccttctctagaaccagcatggataaaggcctacaaggcgctctaaaaaagaagatctaaaaactataaaaaaaataattataaaaatatccccgtggataagtggataaccccaagggaagttttttcaggcatcgtgtgtaagcagaatatataagtgctgttccctggtgcttcctcgctcactcgagggcttcgccgtcgctcgactgcggcgagcctactggctgtaaaaggacagaccacatcatggttctgtgttcattaggttgttctgtccattgctgacataatccgctccacttcaacgtaacaccgcacgaagatttctattgttcctgaaggcatattcaaatcgttttcgttaccgcttgcaggcatcatgacagaacactacttcctataaacgctacacaggctcctgagattaataatgcggatctctacgataatgggagattttcccgactgtttcgttcgcttctcagtggataacagccagcttctctgtttaacagacaaaaacagcatatccactcagttccacatttccatataaaggccaaggcatttattctcaggataattgtttcagcatcgcaaccgcatcagactccggcatcgcaaactgcacccggtgccgggcagccacatccagcgcaaaaaccttcgtgtagacttccgttgaactgatggacttatgtcccatcaggctttgcagaactttcagcggtataccggcatacagcatgtgcatcgcataggaatggcggaacgtatgtggtgtgaccggaacagagaacgtcacaccgtcagcagcagcggcggcaaccgcctccccaatccaggtcctgaccgttctgtccgtcacttcccagatccgcgctttctctgtccttcctgtgcgacggttacgccgctccatgagcttatcgcgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgatgataagctgtcaaacatgagaattggtcgagtttaaacatgcatccttaataacttcgtatagggtaggctatacgaacggtaaggcggatctaaactgaaagttgcagctttagatggcaaggaaacttgggtttcaggcatagaaagcctggctcactatagcagcccatgtttgttccagggtgggggaaaggcacgtgcccttagaaaacttagctgcaaaaattctattgtgttgggagagcctctatatctaaagggatccagacatgataagatacattgatgagtttggacaaaccacaactagaatgcagtgaaaaaaatgctttatttgtgaaatttgtgatgctattgctttatttgtaaccattataagctgcaataaacaagttgcccgggcccgagtgcgtgggcatgacaatcgtgccctggggaccaacacaatccagaagggcctgaatcactgcgaccggccctcccgcgacccagccgagcgagcttaaattcttaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcatggtggcgaccggtagcgctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaaacgtgccggcctttcctcacaatacaaatgttactaacgtctgccctctggagacctgctatgtggctagacgtatggcctacccaagacgttgggggtctcgggtaggccatgattcttccaggcataggttacaaccagtcactgctatcaagcctactcagttcccaacgcagcacataccccccgcctctcctgccatgaggacttatggcagtgtttactgttctgcttttactcttggaccaggctgtcattctatcagaataacaggggaagcaaggccccctgcttcagcgggacacgtgtttctagaatctcggagccaataactacctgcccctctaatctgtatgctgcatgaaaaaccacatacacgtgatgtaagtttagccagtttattgttacaccaatgccccgaaagtctccccctgtccctttgggtctcaggacccagccctggagctcgggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggcccgctgccccgctccggcggggggtggccggctgcagccgggtccggggttccggccctggagctcggggggcggccgggtggcccaccgggtccgctgggtccgctgccccgctccggcgggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcgggggacggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgagtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggtccgctgccccgctccggcggggggtggccggctgcagccgggtccggggttccggccctggagctcggggggcggccgggtggcccaccgggtccgctgggcccgctgccccgctccggcggggatgggggtgcgctcccaggccggaccctggtgccaggcagggaccccgcgccacccgcttcatggggggggaggccgccgcaaggacgccgggccggctgggaggtgtgcaccccccgagcgtctggacgacgctggcgagccgggccagctcgccttcttttatcctctttttggggtttctgtgcaataccttaaggtttgctcaggtgtggggggcttctcattggttaattcaggtgtgtgattttagcccgttgggttacattaaggtgtgtaaccaggtgggtggtacctggaggtcattctattgggataacgagaggaggaggggctagaggcccgcgagatttggggtaggcggagcctcaggagggtcccctccatagggttgaaccaggagggggaggagtgggctccgccccgatatacctagtgggtggagcctagaggtaggtctccatagggttccattatcctggaggtatcctaagctccgcccctatataccaggtgggtggagctaggtaggattcagctaggttcctactggggtacccccctaccctaccttaaggtgcgccacccttcctccttccgttttaatggtagaataacctataggttattaacctagtggtggaatagggtattgcagctgggtatatacctataggtatatagaacctagaggaagggaaccctatagtgtaatccctcccccccctacccccccctcccttacggttgcctgagcccatcccccaccccagcaccccggggtgacgtggccccccgcgtgccttactgacttgtcacctttgcacatttggtcagctgaccgatgctcgccacttcctgggtcatgacctggcctgtgccttgtcccgtggacaatgtccctccagcgtggtggctgcctttgggatgcatcactttgagccactaagcccccgttgctcgccttgcctgcctcaccatgacacactaagcccctgctaatccatgagccccgcctttaggaagcaccacgtcccggggacggaagctggattttggccagtcttcaatttttgggagtggttttgtgtgagccggaagttggcaatggggtgagggcggcgctggttaagctgacgacctcccaaggtctctcaccctgggtacacaggtggggcggcagcctctaactttggctgtggcctctatttcctccctttcctagccagggccatgtgttcctgcatgtctacttgcctcctgtggtggcagagcttggccctgtgcccaacccccgccttgggagcctgtaggggccaacacccttggtttgtttgtgttcctgtttgctggcaacttactggcagccgagcagattctaatgggcgcccgccttctttctctcttgttttattaatagaatctcagccaggacctatacctgagacttcaaagtctggtcctgggttctgagacccccaagatttgtcatgcacacctgcacacctgttggtattgggtttctattcttgagtgtgaaagtttgtaaaaaaattcataaaatgtcactaattcctcttacctgtttagggtattgtgcaattcttcagcctgcctattttcaatttgcctaaggtggcaatttaagatgtggttaattaaccattttcctgtctgacaccactgcatgggcaaccgggttccatggcacatttagagataaacatagatgtcttgtcttgctcatgtgcagaggagggggtgttggtgtgcaatatagtttctggattccaaattgagttgggggtgctattttcactatggaattaaattactgacattagacagtggacacccggctatatgtggggatgtctgtggcttgtcatttcctcttagaaggtaatcccccatcttaacttccctttaaattgtgatgcaagccctgggttatttatagaatgattatctaggtttgatagtctgaaggctgggcagagaatgtttgtaatttttattcaccttctttaccccccacgagtatccagttctagaagatcctacgaggccgagacgggcagccggagcttgtggtactgtccctccggcaggtggagtgggacacagttagagaacattagtcctctggtccctatctccacccgccaggcctgtgtgtcagtttgcagggccatcctcgcactcaggtggactggctaggcacccttctgaagtatctggcggtgactgtcacctggttcttgagagagtccataaaatggctgaagctccaggcgtatagtataatgagcaacagggccaaacaggcggcggggcctgggtagtagcgggcaacgagagactctgtgcaatcaaaccccaggctcccggcctcacccaggaagagcagcggcagggacagcataaaccaggagaaggcgcaaatgagtccggtgaaggtgacgttgcatatcaggcgcggcttccttccgaattttgtgcgcaaaagtttccagatgatgataactgtgaggaggacgatcaggactgccgccagtaggtagcagccggctttcagtccttggacggccgtgtgcatgcctttggtggggccttccctgcacatgttggggcctctgttgagattggcgtcggggcccatggtaatgaggaggatgataatcagcaggagtaccagacaaaacacgcccatcaggtacaggcacacatttctgtgggaggttctcttgggcgttcggctgaacaatgctagggtcttctccaacgccatacccaagtgagtccatacggagcacatcaggcccaagaacatcatgttctgggtcaaaaggcagagaccggtagacgagaactcctgaatcatttttcccagcacccagagcagcagttctatgagaagagctatcagccagacatccattcggtgaaccaattttcttacaaagatgataaacaagatgccagccagtgttagcagaatcagcaggacgagcagcaggcttgtcatgccgctgaggaaggcgctgtaggatttagtgcacgcatcttccgttgcattgacggaagtcatgttggccaccagggtccccacggtggacccgggggccatggtggagagcat

SEQ ID NO: 21 - DNA, 3409 bp, artificial sequence
  nucleotide coordinates #176,332 to #179,740 of Delta miR (4027)
aatccctctcgccttgtttctcagagagcatttcaggccggttttgcagtcgctgctgcagctatggggtccctagaaatggtgccaatgggcgcgggtccccctagccccggcggggatccggatgggtacgatggcggaaacaactcccaatatccatctgcttctggctcttctgggaacacccccaccccaccgaacgatgaggaacgtgaatctaatgaagagcccccaccgccttatgaggacccatattggggcaatggcgaccgtcactcggactatcaaccactaggaacccaagatcaaagtctgtacttgggattgcaacacgacgggaatgacgggctccctccccctccctactctccacgggatgactcatctcaacacatatacgaagaagcgggcagaggaaggtaagagtgccatctatctgtacttttatttattgcatcacaagtcacatcaataataagggcgccatctagcgggagatgttatccacaccatcccaattcacatctcagggacaacaggtcaaagttctttgttgacacccccagcgctggctccagggggtggaagcgttggatgcagtcctccgcatcggggcggacgcctcctcccaacgcgtttctgcggatcagtcgctggctggtgggcatcggagtcggtgggcggtcctccacggggacacgctccttcttggccttgttctttgaccttttggacattcttctgaaggaacggcggagagtagcgtagaatccagccagtggtctacccggtcgcatggtggcttcttagatgaggagcaggcataaaagtccaaacaggacacagagtaccaccaggagtagtcttagtctgctgacgtctgggtcctcggggcaggggtggctaggcctggtctccgtagaagagccgggcaggccgcaggcagaggactgctgctctagcaaagcacgctccaggacgtgtaccatctcgagagtgaggcacagctgttttcgtggacttttatacagtaaggacaaggaaagaaggccagaggaatgtggaaagatgagcgaggacaggtgtggaggttttgggctagctcttagtttctgggtgtgagagagggattaaagtgcttatgcgcaaagaatgtgtcaacaacaggtgttcctgcctctgctggcatgagttaggtgtggcttgggctgaatccaaatgtgtattggcacaagatggaaagcaaagttgctggagttactgggtgggagacagggatgtatgtggtcccccgctggtatgccagtaccctgtggaagtaaggggcctcatctgcctggtagttgtgttgtgcagaggtctgatgtgtgtaggaggggtgggttcaacgcaggggcgttggtggcggagtctggcaacgcccgggtccttgctacctgtgtggtgtgttaagggctgggtaaaggtgtctgccaattctcgcatgtcctcctttccccttgttttgaaatagaatatgaatgtggcttttcagcctagacagacagtgtggctaagggagtgtgtgccagttaaggtgattagctaaggcattcccagtaaatggagggagagtcagtcaggcaagcctatgacatggtaatgcctagaagtaaagaaaggttagtcatagtagcttagctgaactgggccgtgggggtcgtcatcatctccaccggaaccagaagaacccaaaagcagcgtaggaaggtgtggatcaccgccgccatggccggaatcatgactatgaccgccgcctccgtctgtcatcaaaggcgggccctggtcacctcctttgttttcaacctcttccgtcaattgtggagggcctccatcatttccagcagagtcgctagggctatgaggcagcgggtcatgtgggccattgtcatcagtgttgtcagggtcctgtgggccattgtcatcagtgttgtcagggtcctgaggcagcgggtcatgtgggccattgtcatcagtgttgtcagggtcctgtgggccattgtcatcagtgttgtcagggtcctgtgggccattgtcaggaccacctccaggtgcgcctaggttttgagagcagagtgggggtccgtcgccggctccactcacgagcaggtggtgtctgccctcgttggagttagagtcagattcatggccagaatcatcggtagcttgttgagggtgcgggagggagtcatcgtggtggtgttcatcactgtgtcgttgtccatggtaatacatccagattaaaatcgccagaaacaggaggagccaaaggagatcaaccaatagagtccaccagttttgttgtagatagagagcaataatgagcaggatgaggtctaggaagaaggctaggaagaaggccaaaagctgccagatggtggcaccaagtcgccagagcatctccaataagtagatccagatacctaagactgcgttgaaaaaagagtgttagggttggaaaagtgggggtgtggtaaataattcctagggaatgttagatcttaccaagtaagcacccgaagatgaacagcacaattccaaggaacaatgcctgtccgtgcaaattccagagagcgatgagcaggagggtgactggggaaagaggagaaagtgcgttagagaaggaagagtaagggaaagggggtgtggggcaaagggtgtaatacttactcatcagtaggagtatacaaagggctccaagtggacagagaaggtctcttctgaagataaagatgatcaaaattataattataagcatgagagcaaaggaatagaggacaaggagggctcctccagtccagtcactcataacgatgtacagccaaaacagtagcgccaagaggaggagaaggagagcaaggcctagggaagaggagagggggggtcctcgagggggccgtcgcgggcccggtgggcccctctcaaggtcgtgttccatcctcagggcagtgtgtcaggagcaaggcagttgaggaaagaagggggcagagcagtgtgagaggcttatgtagggcggctacgtcagagtaacgcgtgtttcttgggatgtaggcccggggggatttgcggggtctgccggaggcagtacgggtacagatttcccgaaagcggcggtgtgtgtgtgcatgtaagcgtagaaaggggaagtagaaagcgtgtgtttgtgttagaaaagcgggtccccggggggcaagctgtgggaatgcggtggccaagtgcaacaggaaatggaaaggcagtgcggcaatcagaagggggagtgcgtagtgttgtgggaagcggcagtgtaatctgcacaaagaggcgcggggcgcgcaacgttgggaggtcgttggcggcaggcgggaggccgtgctttaggggggttcag

SEQ ID NO: 22 - DNA, 2097 bp, artificial sequence
  EB-VLP producer genome p6507.8 from nucleotide coordinates # 183,075 to #185,171
aatccctctcgccttgtttctcagagagcatttcaggccggttttgcagtcgctgctgcagctatggggtccctagaaatggtgccaatgggcgcgggtccccctagccccggcggggatccggatgggtacgatggcggaaacaactcccaatatccatctgcttctggctcttctgggaacacccccaccccaccgaacgatgaggaacgtgaatctaatgaagagcccccaccgccttatgaggacccatattggggcaatggcgaccgtcactcggactatcaaccactaggaacccaagatcaaagtctgtacttgggattgcaacacgacgggaatgacgggctccctccccctccctactctccacgggatgactcatctcaacacatatacgaagaagcgggcagaggaaggtaagagtgccatctatctgtacttttatttattgcatcacaagtcacatcaataataagggcgccatctagcgggagatgttatccacaccatcccaattcacatctcagggacaacaggtcaaagttctttgttgacacccccagcgctggctccagggggtggaagcgttggatgcagtcctccgcatcggggcggacgcctcctcccaacgcgtttctgcggatcagtcgctggctggtgggcatcggagtcggtgggcggtcctccacggggacacgctccttcttggccttgttctttgaccttttggacattcttctgaaggaacggcggagagtagcgtagaatccagccagtggtctacccggtcgcatggtggcttcttagatgaggagcaggcataaaagtccaaacaggacacagagtaccaccaggagtagtcttagtctgctgacgtctgggtcctcggggcaggggtggctaggcctggtctccgtagaagagccgggcaggccgcaggcagaggactgctgctctagcaaagcacgctccaggacgtgtaccatctcgagagtgaggcacagctgttttcgtggacttttatacagtaaggacaaggaaagaaggccagaggaatgtggaaagatgagcgaggacaggtgtggaggttttgggctagctcttagtttctgggtgtgagagagggattaaagtgcttatgcgcaaagaatgtgtcaacaacaggtgttcctgcctctgctggcatgagttaggtgtggcttgggctgaatccaaatgtgtattggcacaagatggaaagcaaagttgctggagttactgggtgggagacagggatgtatgtggtcccccgctggtatgccagtaccctgtggaagtaaggggcctcatctgcctggtagttgtgttgtgcagaggtctgatgtgtgtaggaggggtgggttcaacgcaggggcgttggtggcggagtctggcaacgcccgggtccttgctacctgtgtggtgtgttaagggctgggtaaaggtgtctgccaattctcgcatgtcctcctttccccttgttttgaaatagaatatgaatgtggcttttcagcctagacagacagtgtggctaagggagtgtgtgccagttaaggtgattagctaaggcattcccagtaaatggagggagagtcagtcaggcaagcctatgacatggtaatgcctagaagtaaagaaaggttacctcagggcagtgtgtcaggagcaaggcagttgaggaaagaagggggcagagcagtgtgagaggcttatgtagggcggctacgtcagagtaacgcgtgtttcttgggatgtaggcccggggggatttgcggggtctgccggaggcagtacgggtacagatttcccgaaagcggcggtgtgtgtgtgcatgtaagcgtagaaaggggaagtagaaagcgtgtgtttgtgttagaaaagcgggtccccggggggcaagctgtgggaatgcggtggccaagtgcaacaggaaatggaaaggcagtgcggcaatcagaagggggagtgcgtagtgttgtgggaagcggcagtgtaatctgcacaaagaggcgcggggcgcgcaacgttgggaggtcgttggcggcaggcgggaggccgtgctttaggggggttcag